(19) United States
(12) Patent Application Publication
(10) Pub. No.: US 2014/0121467 A1
(43) Pub. Date: May 1, 2014

(54) METHODS AND APPARATUS FOR SIMULTANEOUS RETRACTION AND DISTRACTION OF BONE AND SOFT TISSUE

(71) Applicant: Invuity, Inc., San Francisco, CA (US)

(72) Inventors: Alex Vayser, Mission Viejo, CA (US); Carl Basamania, Shoreline, WA (US)

(73) Assignee: Invuity, Inc., San Francisco, CA (US)

(21) Appl. No.: 14/060,848

(22) Filed: Oct. 23, 2013

Related U.S. Application Data
(60) Provisional application No. 61/720,839, filed on Oct. 31, 2012.

(51) Int. Cl. A61B 17/02

(52) U.S. Cl. CPC A61B 17/025; A61B 17/0218
USPC 600/214; 600/201; 600/245; 600/228; 600/226; 600/235

(57) ABSTRACT

A surgical access system includes an access device having a tubular shape and a channel extending through the device that is sized to receive a surgical instrument. The outer surface of the device defines a support section and access section having an aperture extending through the wall of the device. The aperture has a wider distal portion and a narrower proximal portion and the surgical instrument may be inserted through the aperture. The access device simultaneously retracts soft tissue and distracts bone when inserted into the surgical site while allowing a surgeon access to the surgical site.

### CROSS-REFERENCE

The present application is a non-provisional of, and claims the benefit of U.S. Provisional Patent Application No. 61/720,839 (Attorney Docket No. 40556-727.101) filed Oct. 31, 2012; the entire contents of which are incorporated herein by reference.

### BACKGROUND OF THE INVENTION

1. Field of the Invention

Providing surgical access to a treatment site remains a challenge, especially given the movement toward smaller incisions and minimally invasive procedures. While the present invention will be described with embodiments primarily directed at shoulder surgery, one of skill in the art will appreciate that this is not intended to be limiting. The devices, systems, and methods described herein may be used for treating shoulder injuries such as the rotator cuff, and they may also be used to treat other parts of the body requiring surgical access.

The human shoulder includes the clavicle, scapula and humerus as well as several muscles, ligaments and tendons. The acromion is a bony process on the scapula that extends laterally over the shoulder joint. The major shoulder joint is the glenohumeral joint where the humerus attaches to the scapula. Other joints in the shoulder include the acromioclavicular and the sternoclavicular joints. The rotator cuff is a group of muscles and their corresponding tendons that stabilize the shoulder including holding the head of the humerus in the glenoid fossa. These muscles arise from the scapula and connect to the head of the humerus.

Rotator cuff tears occur typically when one of the tendons of the rotator cuff muscles tear. They represent one of the most common orthopedic injuries in the United States. According to the American Academy of Orthopaedic Surgeons, over 5 million physician visits were attributed to rotator cuff problems between 1998 and 2004. At the present time, there are three basic approaches to rotator cuff surgery: open repair, mini-open repair and arthroscopic repair. There are advantages and disadvantages to each of these techniques. The traditional open technique involves cutting the deltoid muscle away from the acromion and then removing bone from the underside of the acromion, in addition to removing bursal tissue. The rotator cuff is then repaired utilizing suture anchors or transosseous sutures and the deltoid is then reattached to the acromion. The primary challenge with this approach is that it is rather invasive and risks compromise of the deltoid muscle if it fails to heal back to the acromion. Being a more extensive surgical approach, it is typically associated with more pain compared with less invasive procedures. The primary advantage of this technique is relatively good exposure to the torn tendon. Furthermore, any size rotator cuff tear can be treated with this approach and there is little in terms of specialized training necessary to perform this technique.

Due to the invasiveness of this procedure, a “mini-open” technique was developed that typically utilizes an arthroscopic examination of the shoulder. Once this is completed, an incision is made over the lateral aspect of the shoulder and the deltoid muscle, instead of being cut away from its attachment on the acromion, is split in line with its fibers. This allows for exposure of the torn tissues but does not compromise the integrity of the deltoid. The primary problem with this approach is exposure. It can be very difficult to see and work on the torn cuff through this approach. In order to gain better exposure, many surgeons utilize devices such as lamina spreaders to distract the humeral head from the acromion. Although this can help with the exposure, these instruments can cause damage to the acromion and the articular surface of the humeral head. It also can be difficult to work around these retractors. In addition to exposure, it can also be difficult to pass and use instruments through the small split in the deltoid. However, it is less invasive than the open technique in that most of the debridement of damaged tissue is performed arthroscopically. The other primary advantage of the mini-open technique is that the cuff can be repaired using suture anchors or a transosseous technique.

With the arthroscopic technique, the entire procedure is performed utilizing multiple small incisions and arthroscopic placement and tying of the sutures to repair the cuff. The theoretical advantage of this technique is that it is minimally invasive and is almost always performed on an outpatient basis. It is assumed that there is less pain associated with this technique. The primary problem with this technique is that there is a rather steep learning curve to become skilled with this approach. The other primary problem is that it can be very difficult to manage the sutures used to repair the cuff. Furthermore, there can be problems with excessive fluid build-up in the soft tissues about the shoulder. Since visualization is critical to using this technique, most surgeons use an arthroscopic pump to distend the joint and subacromial space. While this can help with the visualization, the fluid can dissect into the soft tissues about the shoulder, particularly the deltoid, and this can limit the time a surgeon has before he has to abandon the technique due to excessive soft tissue distention. It also can only be performed using suture anchors. In addition to being a considerable expense which typically cannot be recovered in an outpatient surgical setting, there are also problems associated with the use of suture anchors. Suture anchors can fail in three ways: anchor pullout, in which the anchor comes out of the bone intact, including the suture; suture breakage, in which the suture fails at the point at which it is connected to the anchor; and eyelet breakage, in which the suture pulls out intact after cutting through the eyelet of the anchor. Furthermore, repairs performed arthroscopically typically have to be performed using weaker suture techniques such as simple or horizontal suture placement in tendon whereas open and mini-open techniques can use stronger grasping suture patterns such as a Mason Allen type stitch. Finally, suture “management” can be very difficult with the arthroscopic technique because the sutures must be passed and retrieved through multiple small cannulas. The sutures can become entangled in these cannulas and becomes an even bigger problem when multiple anchors or anchors with multiple sutures are used.

Therefore, given the challenges of accessing the shoulder joint, new devices, systems, and methods are needed. Such devices, systems and methods preferably allow for a minimal incision, circumferential and simultaneous distraction and retraction with a single device and significant improvement in visualization utilizing illumination technology. The approach should be comparable or superior to open and arthroscopic techniques. It is preferably performed through much smaller incisions compared to traditional open techniques and without fluid distention of the surgical site as required in arthroscopic repairs. In addition, suture management may be addressed by passing all the sutures through a single portal. Such approach is also preferably “minimally invasive”, so that the technique can easily be performed in an outpatient setting. Furthermore, the technique preferably allows the surgeon to perform the repair utilizing suture anchor or transosseous techniques and does not restrict the surgeon to only simple or horizontal suture patterns as is the case with arthroscopic techniques. Such technique should also have little, if any, “learning curve” effect with this technique. At least some of these objectives will be met by the devices, systems and methods described below.

2. Description of the Background Art

Patents and publications which are related to surgical access include US Patent Publication Nos. 2009/0287061; 2005/0171551; and U.S. Pat. Nos. 7,811,303; 7,976,463.

### SUMMARY OF THE INVENTION

The present invention relates to medical devices, systems, and methods. More particularly, the present invention relates to surgical access devices and their use, including but not limited to those used for rotator cuff surgery.

In a first aspect of the present invention, a system for accessing a surgical site in a patient comprises an access device configured for insertion into the surgical site. The access device has a tubular shape with a wall having a wall thickness, a proximal end, a distal end, a channel extending between the proximal and distal ends, and an outer surface circumferentially disposed around the access device. The outer surface defines a support section and the outer surface also defines an access section having an aperture extending through the wall. The channel is sized to receive a surgical instrument, and the aperture comprises a distal portion and a proximal portion. The distal portion is adjacent the distal end of the access device, and the proximal portion is proximal of the distal portion. The distal portion has a width that is wider than the width of the proximal portion. The channel and the proximal and distal portions of the aperture are sized to receive the surgical instrument, and the access device is configured to simultaneously retract soft tissue and distract bone when inserted into the surgical site while allowing the surgical instrument access to the surgical site.

The access device may form a cylindrical tube or an oval, elliptical, square, diamond, or round tube. The access device may have a distal end that is chamfered or tapered. The wall in the support section may extend further distally than the wall in the access section. The system may further comprise a plurality of increasingly sized tissue dilators slidably disposed over one another. The plurality of tissue dilators may be sized for slidably positioning in the channel of the access device. A tissue dilator may comprise an actuatable dilator mechanism having a plurality of arms movably coupled to an outer frame, and an engagement element coupled to each of the arms. Actuation of the arms may move the engagement elements inward or outward relative to one another. And, the engagement elements may retract tissue and distract bone when engaged therewith and actuated. The access device may have two separate blades that are engaged with one another to form the tubular shape.

An illumination element such as fiber optics or an optical waveguide may be coupled with the access device, and the illumination element may illuminate the surgical site. The access device may comprise a channel for receiving the illumination element. In some embodiments, the access device may be formed from an optical material such that the access device itself is also an optical waveguide configured to illuminate the surgical site. In other embodiments, the access device may have an optical portion that is for illumination, and also a non-optical portion. The optical portion may be fabricated from any optical material and the non-optical portion may be fabricated from metals, polymers, or other materials known in the art.

The access device may comprise a flanged region adjacent the proximal end. The flanged region may be configured to limit insertion depth of the access device into the surgical site. The access device may also comprise one or more suture management features adjacent the proximal end, and that may be configured to prevent entanglement of sutures. The system may further comprise one or more sutures, and the suture management features may comprise slotted regions adjacent the proximal end of the access device.

The access device may further comprise an engagement element for coupling with a table mounted arm or wall mounted arm. The outer surface of the access device may define an access window that is disposed in the support section of the access device, and that is sized to receive the surgical instrument thereby providing greater access to the surgical site. The access window in the support section of the device may extend all the way to the proximal end of the access device such that the access window is open on its proximal end. Furthermore, the access device may serve as an electrode, or may have an electrode coupled to it for neurostimulating tissue adjacent the treatment site. Thus, while the access device is being inserted into tissue, the electrode may be energized and adjacent nerves can be localized. This ensures that nerves are not inadvertently damaged during insertion of the access device.

An obturator may also be included in the access system, and the obturator may be disposed in the channel of the access device. The system may further comprise a grasping element such as a handle that is releasably attached to the access device. The grasping element may have a plurality of wings for grasping by an operator. The system may also comprise a dilator and a locking mechanism. The dilator may be disposed in the channel and also disposed in the obturator. The locking mechanism may be used to releasably lock the dilator with the obturator.

In another aspect of the present invention, a method for accessing a surgical site through an incision comprises providing an access device having a proximal end, a distal end, a channel extending therebetween, and an outer surface that defines a support section and an aperture. The access device is inserted into the incision and advanced distally toward the surgical site so as to simultaneously retract soft tissue and to distract bones adjacent the surgical site. A surgical instrument is inserted into the channel of the access device and advanced toward the aperture. The target tissue in the treatment site is treated with the surgical instrument while the access device simultaneously retracts soft tissue and distracts bones. The soft tissue may include the deltoid muscle and the bones may include the acromion and the humerus.

The surgical site may be a rotator cuff in a patient's shoulder. The target tissue may comprise a tendon or muscle. Retracting the soft tissue may comprise retracting the deltoid muscle and distracting the bones may comprise distracting the acromion away from the humeral head. The method may further comprise dilating the incision with one or more increasingly sized tissue dilators that are slidably disposed over one another. Dilating the incision may comprise slidably inserting the one or more tissue dilators into the channel of the access device.

The method may also comprise illuminating the surgical site with light. The light may be emitted from an optical waveguide that is coupled with the access device. The access device may be formed from an optical material such that the access device is also an optical waveguide, and the light is emitted therefrom.

The access device may comprise a plurality of suture management features adjacent the proximal end thereof, and the method may further comprise engaging one or more of the sutures with the suture management features so as to reduce to eliminate suture entanglement. The access device may comprise two splitable blades and the method may further comprise separating the two blades away from one another. The method may further comprise manipulating a grasping element such as a handle that is coupled with the access device in order to rotate or move the access device. The method may further comprise inserting an obturator in the access device, or inserting a dilator into the access device and releasably coupling the access device with the dilator.

In the embodiments disclosed herein, a non-fiber optic optical waveguide is preferably used to illuminate the surgical field. However, one of skill in the art will appreciate that other illuminators may also be used, including a fiber optic, LED, or other light sources.

These and other aspects and advantages of the invention are evident in the description which follows and in the accompanying drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B illustrate basic shoulder anatomy.

FIGS. 2A-2C illustrate an exemplary embodiment of a surgical access system.

FIGS. 3A-3B illustrate various patient positions.

FIGS. 4A-4B illustrate use of an arthroscope in the shoulder.

FIG. 5 illustrates surgical portal positions in a shoulder.

FIG. 6 shows an arthroscope advanced into the shoulder joint.

FIGS. 7A-7B illustrate incision location in a shoulder during rotator cuff repair.

FIGS. 8A-8H illustrate an exemplary method of retracting tissue and distracting bone with a surgical access system.

FIGS. 9A-9C illustrate another exemplary embodiment of an access device.

FIG. 10 is still another exemplary embodiment of an access device.

FIGS. 11A-11E illustrate still another exemplary embodiment of an access device.

FIGS. 12A-12D illustrate another exemplary embodiment of an access device.

FIGS. 13A-13B illustrate exemplary embodiments of an access device.

FIGS. 14A-14E illustrate an exemplary illuminator.

FIGS. 15A-15J illustrate a tubular shaped illuminator.

FIGS. 16A-16B illustrate an alternative embodiment of a dilator.

FIGS. 17A-17C illustrate another exemplary embodiment of an access device.

FIGS. 18A-18D illustrate another exemplary embodiment of a surgical access system.

### DETAILED DESCRIPTION OF THE INVENTION

Exemplary embodiments of the present devices, systems, and methods will be described with respect to treatment of a shoulder injury such as a torn rotator cuff. This is not intended to be limiting and one of skill in the art will appreciate that the devices, systems, and methods described herein may be used to treat other portions of the body.

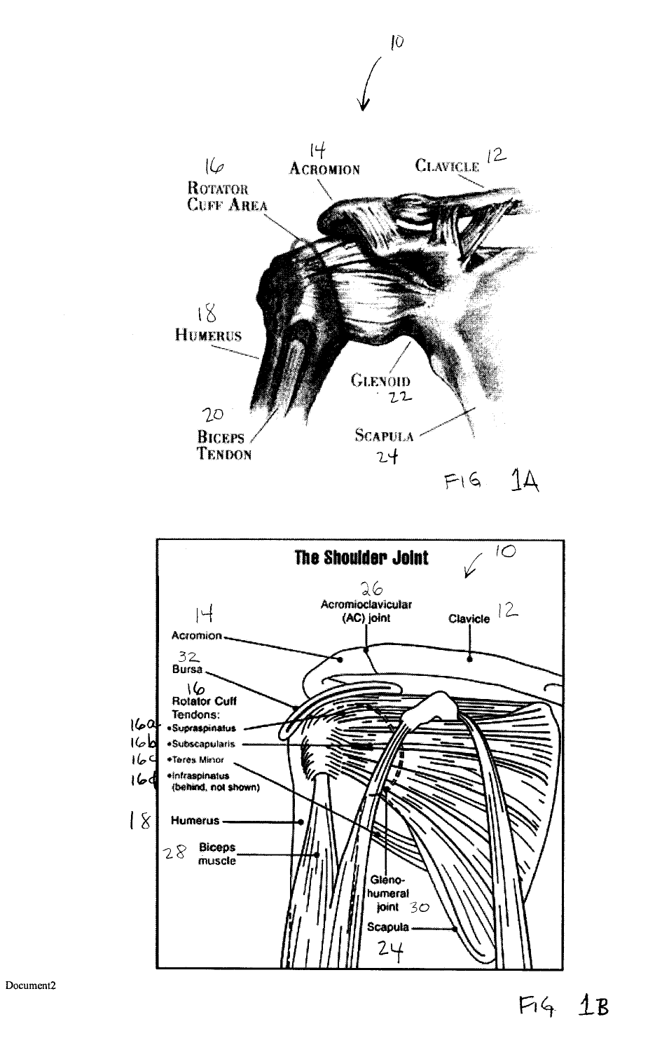

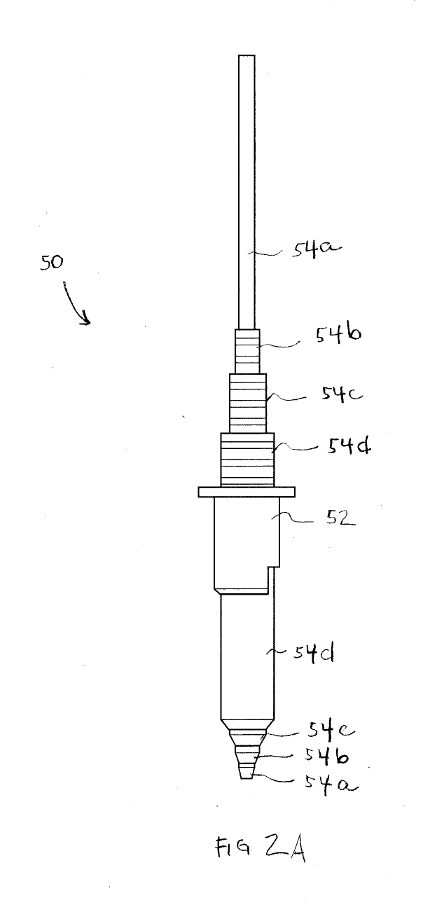

FIGS. 1A-1B illustrate basic shoulder anatomy. The shoulder 10 includes the clavicle (collar bone) 12, scapula (shoulder blade) 24, and humerus (upper arm bone) 18. The acromion 14 is a bony process that extends from the scapula laterally over the shoulder joint. The glenoid 22 is a shallow cavity that forms the socket for receiving the head of the humerus. The biceps tendon couples the biceps muscle to the shoulder. The rotator cuff 16 is a group of muscles and their tendons that stabilize the shoulder including holding the head of the humerus into the glenoid. FIG. 1B illustrates additional details of the shoulder 10 including the acromioclavicular joint 26 which joins the acromion with the clavicle. The bursa 32 is a membrane below the acromion 14. The rotator cuff includes the supraspinatus tendon 16a, the subscapularis tendon 16b, the teres minor tendon 16c, and the infraspinatus tendon 16d. The gleno-humeral joint 30 is also more clearly visible in FIG. 1B showing the humerus head in the glenoid.

In a torn rotator cuff injury, one or more tendons of the four tendons are torn and must be reattached to bone. Typically, the tear occurs at humeral head under the acromion. Thus surgical repair requires access to the humeral head and preferably the acromion is distracted away while adjacent soft tissue is also retracted away in order to provide access to the surgical field.

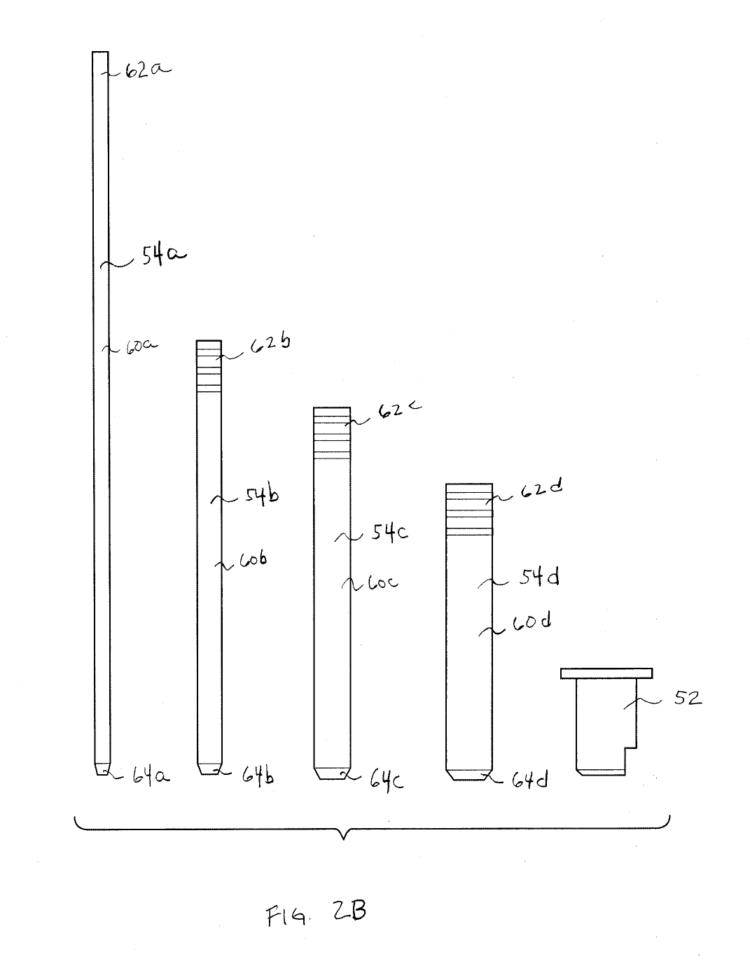

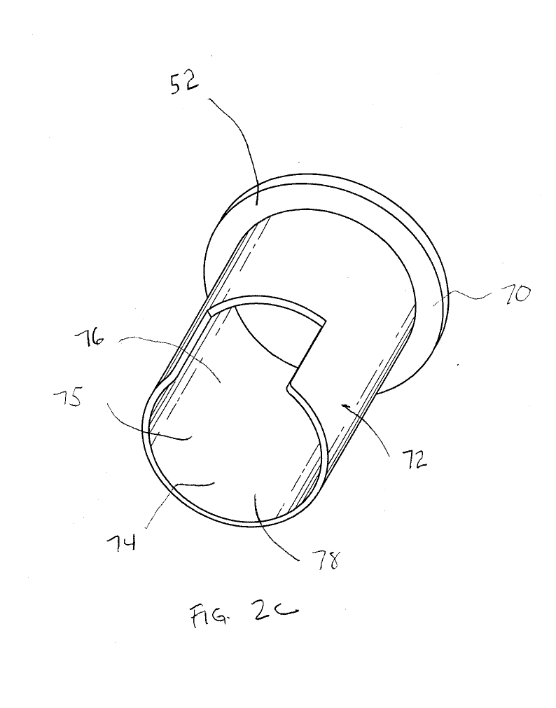

Access System. FIG. 2A illustrates an exemplary embodiment of a system 50 for accessing and allowing repair of a rotator cuff or other surgical treatment site. The repair system 50 includes a series of increasing diameter tissue dilators 54a, 54b, 54c, 54d, and an access device 52. The access device 52 is stacked on top of the largest diameter tissue dilator 54d which is in turn stacked on top of the next smaller tissue dilator 54c, and so on until all the dilators are stacked together with the access device.

FIG. 2B illustrates the tissue dilators and access device once they have been de-stacked. Each tissue dilator 54a, 54b, 54c, 54d includes an elongate shaft 60a, 60b, 60c, 60d with a proximal end and a distal end. The shafts are preferably cylindrically shaped. The proximal end may be textured 62a, 62b, 62c, 62d, knurled, or otherwise formed to facilitate grasping and manipulation by a surgeon. Additionally, the proximal end of the tissue dilators may be color coded, marked or have other indicia on them to indicate the size of the dilator (not illustrated). The distal tip of the tissue dilators may also be pointed 64a, 64b, 64c, 64d, chamfered or otherwise shaped to allow penetration and advancement through the incision and into the shoulder tissue.

FIG. 2C illustrates a perspective view of an exemplary embodiment of the access device 52. The access device is preferably a round tube 72 having a channel 75 extending therethrough, where the outer wall 74 forms a support surface 78 and also defines an aperture 76 or access section. The channel is sized to accommodate various surgical instruments which are inserted into the channel and which may exit the access device through the aperture 76 which is positioned adjacent the treatment site. The flanged region 70 provides a stop to prevent the access device from being advanced too far into the incision. The support surface 78 prevents soft tissue and bone from collapsing inward. Additionally, other features such as suture management features, holding features, lighting features, etc. may be coupled with the flange and are discussed below. Other geometries of the access device, the aperture, and other parts of the access device are possible and these exemplary configurations are not intended to be limiting. Other exemplary embodiments of the access device are described in this specification.

The tissue dilators allow an incision size to be increased by inserting the progressively increasing sized dilators into the incision. The dilators also expand tissue and distract bones deeper in the shoulder as they are advanced distally. Once the last dilator has been inserted into the incision, the access device 52 may be advanced into the incision to simultaneously maintain retraction of the tissue and also distract the bones after the tissue dilators have been removed. Additional details are provided below.

Other Dilators:

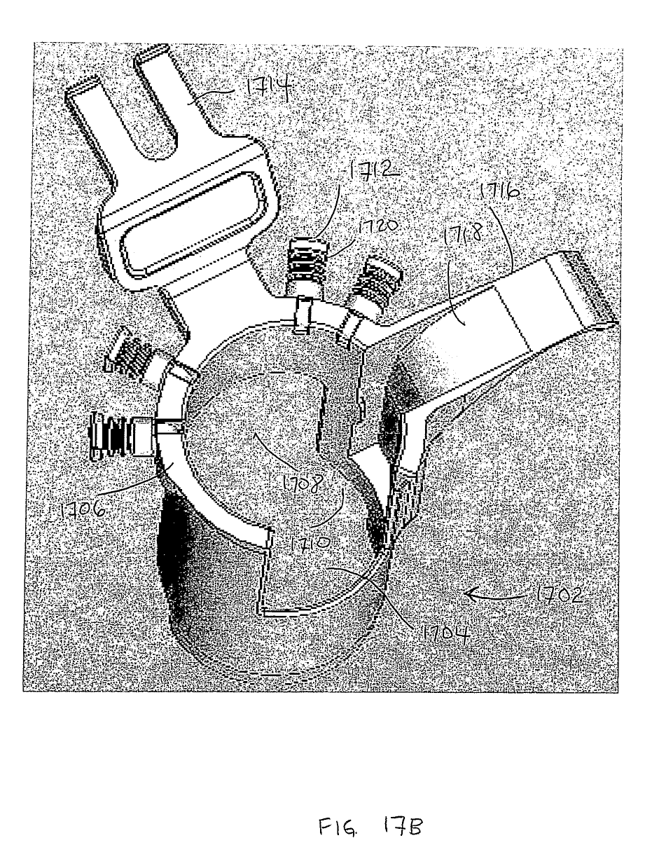

Instead of using the tubular dilators previously described above in FIGS. 2A-2B, other instruments may also be used to dilate tissue and/or distract bone. For example, FIGS. 16A-16B illustrate front and side views of an exemplary embodiment of an actuator mechanism 1602 which includes a frame formed into an outer ring 1604, arms 1606, engagement elements 1610, and actuator elements 1608. In this embodiment, three arms 1606 are movably coupled to ring 1604 and operatively coupled with actuator elements 1608. Movement of the actuator elements 1608, here pins around the outer circumference of ring 1604 move the arms 1606 inward and outward relative to the center of ring 1604 depending the direction that actuator elements 1608 are moved. As the arms 1606 move inward and outward, engagement elements 1610 open and close relative to one another. Thus, in use, the actuator mechanism may be positioned adjacent a surgical incision and the engagement elements 1610 (here pins) may be placed into the incision in engagement with tissue and optionally bone. As actuator elements 1608 are then actuated, arms 1606 will move in an arc inward or outward relative to one another and in the same plane, and thus pins 1610 will also open or close. As the pins open relative to one another, they will engage tissue and retract the tissue. If the pins are also engaged with bone, they will distract the bone simultaneously. Once the incision has been opened to the desired size, the mechanism may be locked into position, and any of the access devices described in this specification may be used to continue the procedure. The actuator mechanism may then be removed once an access device has been placed in the incision. The actuator mechanism 1602 is similar to other self-centering mechanisms known in the art. Any number of arms 1606 may be used and thus the three arms described is not intended to be limiting. Additionally, in this embodiment, the arms are all the same length, however one of skill in the art will appreciate that arm length may be adjusted as required, therefore arm length may not be the same for all the arms.

Other Access Devices: In addition to the access device described above, the systems and methods may include alternative embodiments of the access device such as those described below. Any of the features of the access devices described below may be substituted or combined with the other access devices described in this specification.

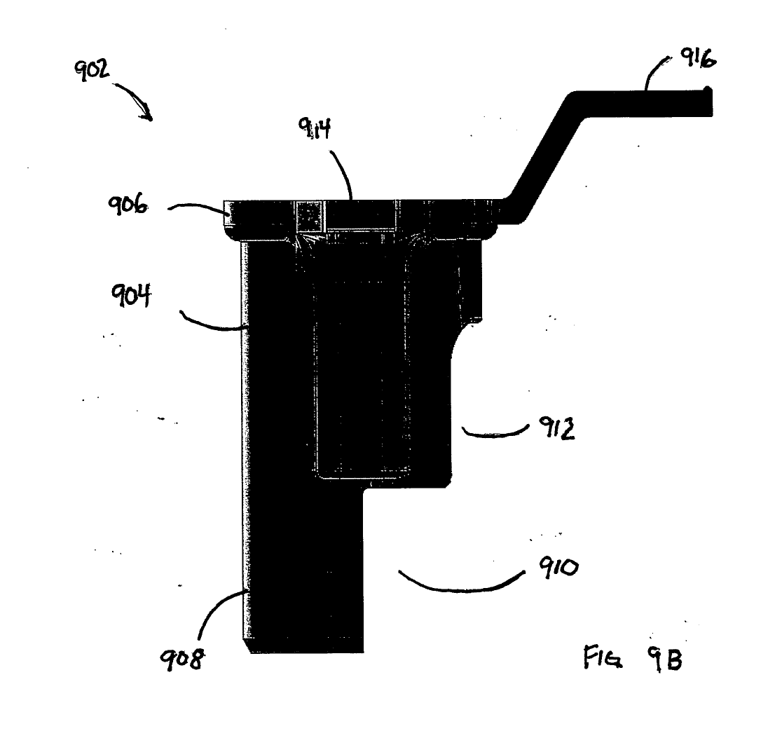

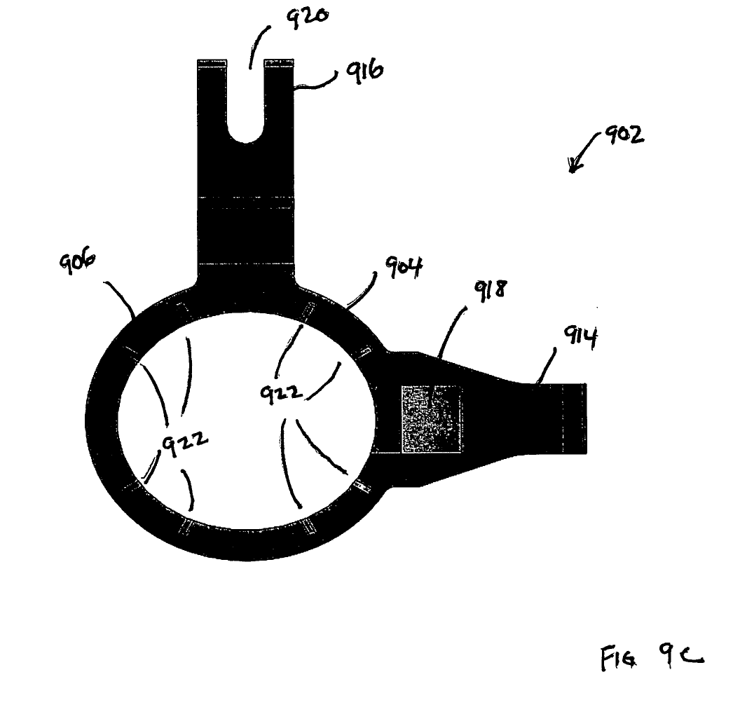

FIGS. 9A-9C illustrate another exemplary embodiment of an access device that may be used with the dilators previously described. FIG. 9A is a side view of the access device, and FIG. 9B is another side view rotated approximately 90 degrees counter clockwise. FIG. 9C is a top view of the device. The access device 902 is preferably an oval shaped tube having a channel extending from the proximal end to the distal end that is sized to receive the surgical instruments used during a rotator cuff repair such as the dilators described herein, as well as other surgical instruments that may be used in a rotator cuff repair or other surgical procedures. The tube is a single tube with a wall 908 that forms an aperture having proximal portion 912 and a distal portion 910. The device includes a flange 906, a coupling element 916, and an illuminator attachment element 914. The flange is used to act as a stop to prevent the access device from being inserted too deep into an incision. Also, the flange may include suture management features such as slots 922 disposed in the flange to keep the sutures from tangling with one another. The coupling element 916 may include a slot 920 for engagement with a standard table or wall mounted arm to help hold the access device in position. The coupling element 916 may be radially and axially offset from the access device. The illuminator attachment element 914 is used in preferred embodiments to receive an optical waveguide or other illumination means (e.g. fiber optics) to provide lighting to the surgical field. It may include a channel for receiving the optical waveguide so that the waveguide is recessed and does not extend into the channel too far taking up valuable space. More details about the illumination are discussed below.

The distal portion 910 of the aperture is defined by removal of about half of the wall 908 from the distal portion of the access device, but may include more or less of the wall. The proximal portion 912 of the aperture is circumferentially narrower than the distal portion and is proximal of the distal portion of the aperture. Thus, the aperture has two rectangular windows axially offset from one another thereby forming a step-like pattern on either side as the aperture transitions from the wider distal portion to the narrower proximal portion. Of course one of skill in the art will appreciate that other window configurations are possible such as elliptical, oval, circular, square, polygonal, etc. shaped windows. The narrower proximal portion of the aperture includes legs 913 on either side that help maintain retraction and distraction of tissue and bone while still allowing access to the repair site. The distal portion of the aperture extends along a longer circumferential arc than the narrower proximal portion of the aperture.

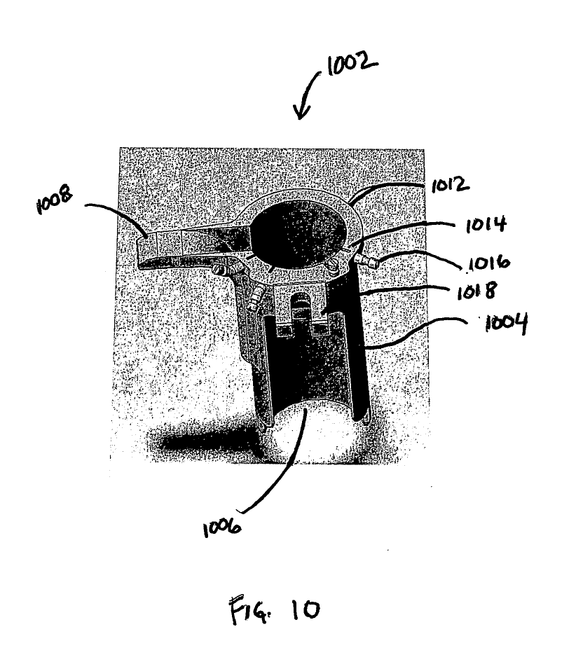

FIG. 10 illustrates another embodiment of an access device 1002 which is similar to the previous embodiment in FIGS. 9A-9C, with the major exception being that it is a round tube and also includes knobs 1016 in addition to slots 1014 for suture management. The sutures may be disposed in the slots or wrapped or tied around the knobs to prevent entanglement and also to allow easy identification. Additionally, the knobs may be used as suture ties for maintaining tension on sutures that are attached to tissue. For example, a suture may be attached to a ligament and the suture is pulled outward to extend the ligament proximally. The suture may then be tied to one of the knobs to maintain the tension and thereby hold the ligament. Thus, the access device not only simultaneously retracts tissue and distracts bone, but may also be used to maintain tension on sutures. Other aspects of the access device 1002 generally take the same form as previously described including the illuminator attachment element 1008, aperture 1006, wall 1004, and coupling element 1018. This embodiment is a single tube.

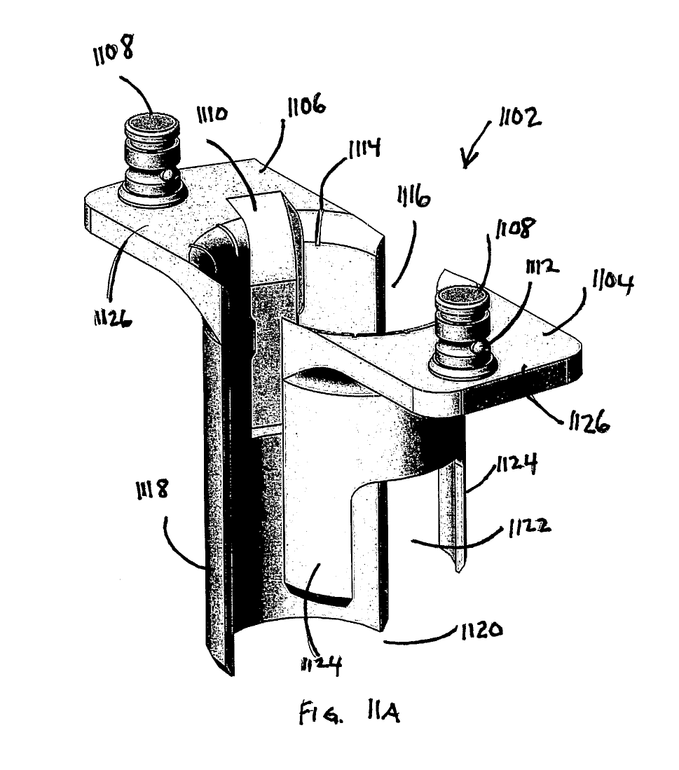

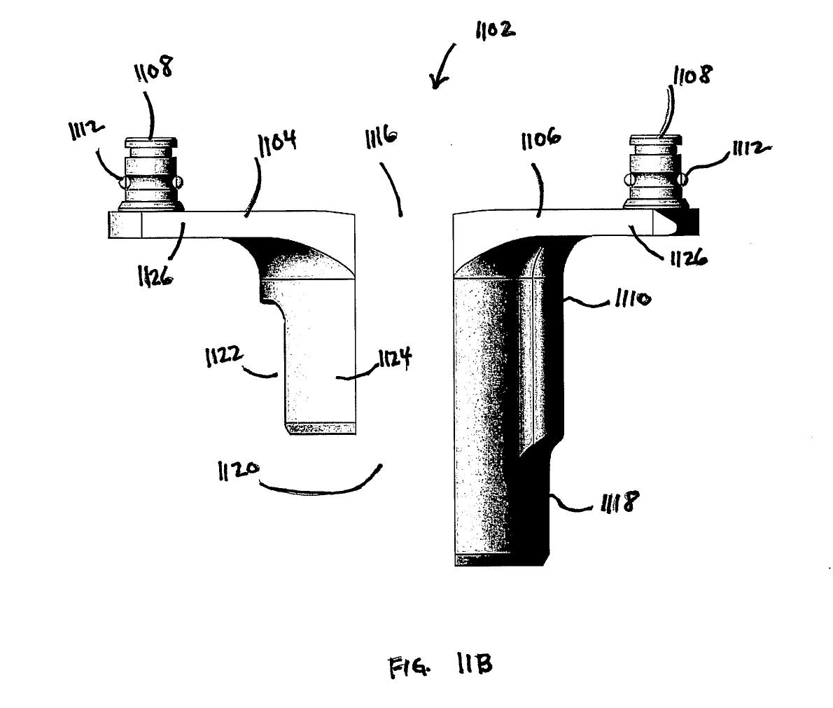

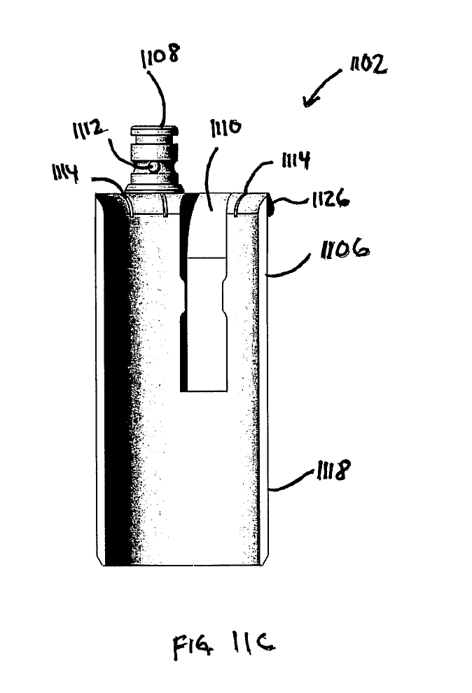

FIGS. 11A-11E illustrate still another exemplary embodiment of an access device that may be used. It is similar to those previously discussed with the major difference being that instead of the access device formed into a single tube, in this embodiment the device is formed from two curved blades engaged with one another to form a tube. The access device 1102 includes two blades 1104, 1106 which are separable from one another. When the two blades engage one another, they form the tube having a channel 1116 that is sized to receive surgical instruments needed for the repair. Other aspects of the device are similar to those previously described above. For example, a flange 1126 on both of the blades prevents excessively deep insertion of the access device into the incision. Also, the flange includes slots 1114 for management of sutures, and an illumination attachment element 1110 allows attachment of a waveguide or other illuminator to the access device for lighting. Pins 1108 with detents 1112 allow attachment of the blades with a retractor frame which can be expanded and collapsed thereby opening and closing the blades relative to one another similar to a Caspar style surgical retractor. The outer wall 1118 of the tube has one side that is longer than the other side, and the shorter side has a portion of the wall removed to form an aperture with a narrow proximal section 1122 and a wider distal section 1120. The narrower proximal portion of the aperture has legs 1124 on either side of the aperture which provide additional support to tissue and bone. The distal portion of the aperture extends along a longer circumferential arc than the narrower proximal portion of the aperture.

FIG. 11B shows a side view of the device in FIG. 11A and highlights the recessed channel that forms the illumination attachment element 1110. This allows a waveguide or other illuminator such as fiber optics to be coupled with the access device 1102 and recessed so as to not take up too much space in the channel 1116.

FIG. 11C illustrates a side view of the inside surface of one half of the access device. This half includes the longer blade having the recessed channel that forms the illumination attachment element 1110. The channel may have a narrow section to provide a snap fit for releasably coupling the waveguide with the blade. FIG. 11D illustrates a side view of the inside surface of the other half of the access device. This half includes the shorter blade having the aperture that allows surgical instruments to access the treatment site. FIG. 11E illustrates a top view of the access device.

The embodiment of FIGS. 11A-11E includes two blades which engage one another in the closed position to form the access device. One of skill in the art will appreciate that any number of blades such as three, four, five, six, or more separatable blades may be used to form the access device and thus the two blade embodiment is not intended to be limiting.

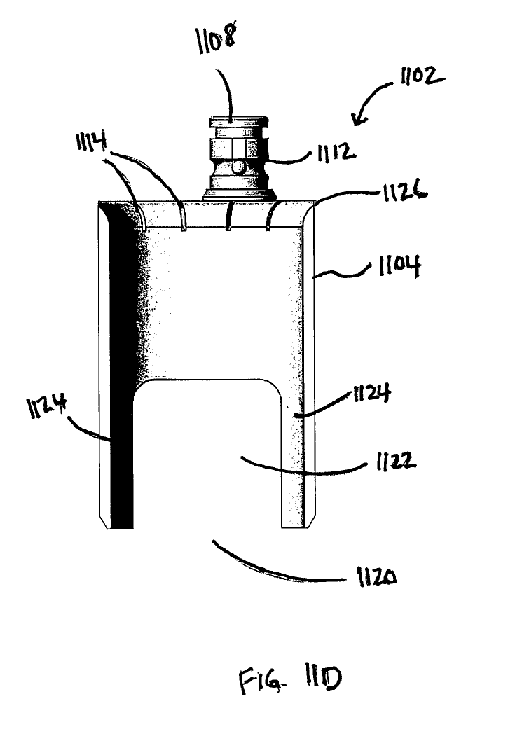

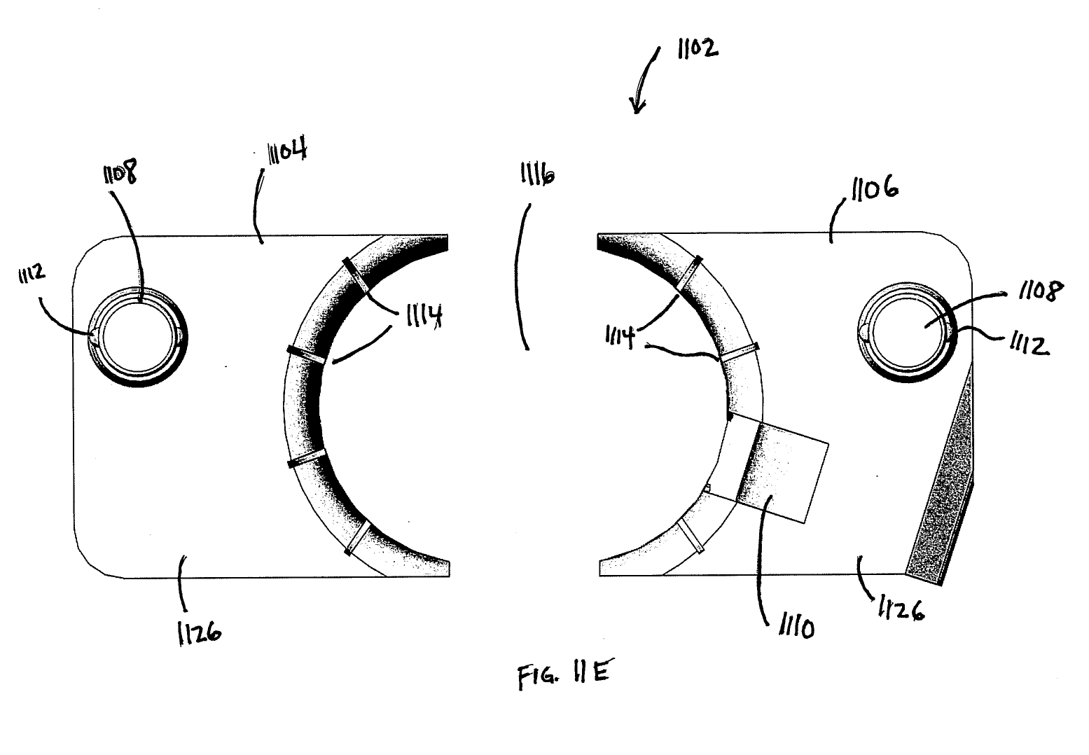

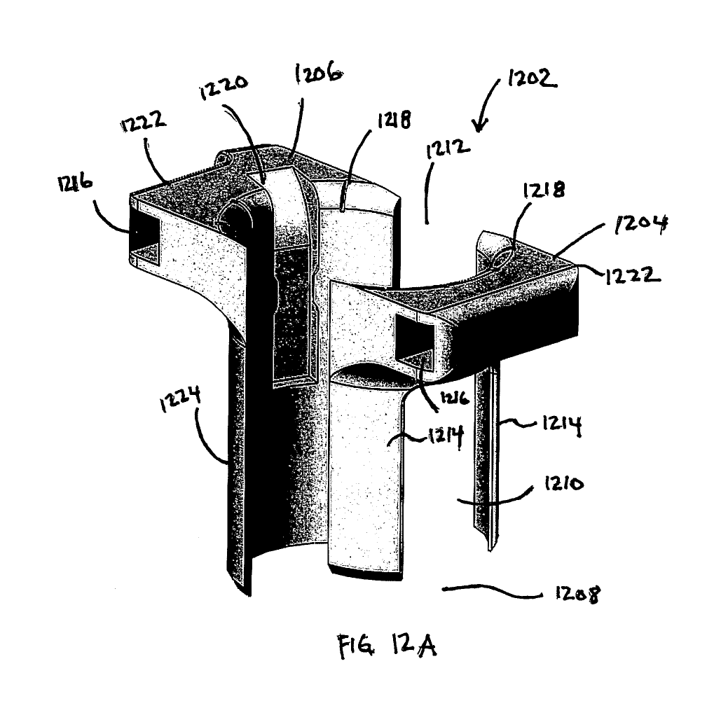

FIGS. 12A-12D illustrate yet another embodiment of an access device 1202. This embodiment is similar to the embodiment of FIGS. 11A-11E with the major difference being that the two blades have channels 1216 for receiving the arms of a retractor frame which can then be expanded and collapsed thereby opening and closing the blades relative to one another. The channels are preferably square channels that extend through the flanged region 1122 of both blades. The square arms of the retractor frame may then be inserted into the channels to support both blades. When the frame is collapsed, both blades engage one another to form a tube with a channel 1212 and when the frame is expanded, the blades move away from one another further retracting tissue and distracting bone away from channel 1212. The tube may be round or oval or other shapes. Other aspects of the access device are generally the same as previous embodiments. For example, the access device 1202 includes two separable blades 1204, 1206. Each has a flange 1222 along its proximal end and may have suture management features such as slots 1218. One blade 1206 is the longer blade where the wall 1224 extends further distally than the shorter blade 1204. The shorter blade has a wall that defines an aperture with a proximal portion 1210 and a distal portion 1208. The proximal portion is narrower than the distal portion. Legs 1214 extend on either side of the proximal portion of the aperture. The longer blade 1206 also may include the illumination attachment element 1220.

FIG. 12B illustrates a side view of the access device 1202. It emphasizes the square channels for engagement with the arms of retractor frame, such as in a McCulloch surgical retractor. Additionally, FIG. 12B illustrates the recessed channel in the illumination attachment element 1220 that allows an illuminator to be coupled with the access device in a low profile configuration to avoid taking up excessive space in the channel 1212. FIG. 12C illustrates a side view of the inside surface of the longer blade 1206. The channel in the illumination attachment element 1220 may have a narrow portion that forms a snap fit for releasably coupling the illuminator with the blade. FIG. 12D is a side view of the inside surface of the shorter blade 1204.

In still other embodiments, the access device may comprise more than two blades that engage one another to form a tube. The plurality of blades may include engagement features such as channels, posts, or other structure to allow the blades to be coupled with a retractor frame or other support structure.

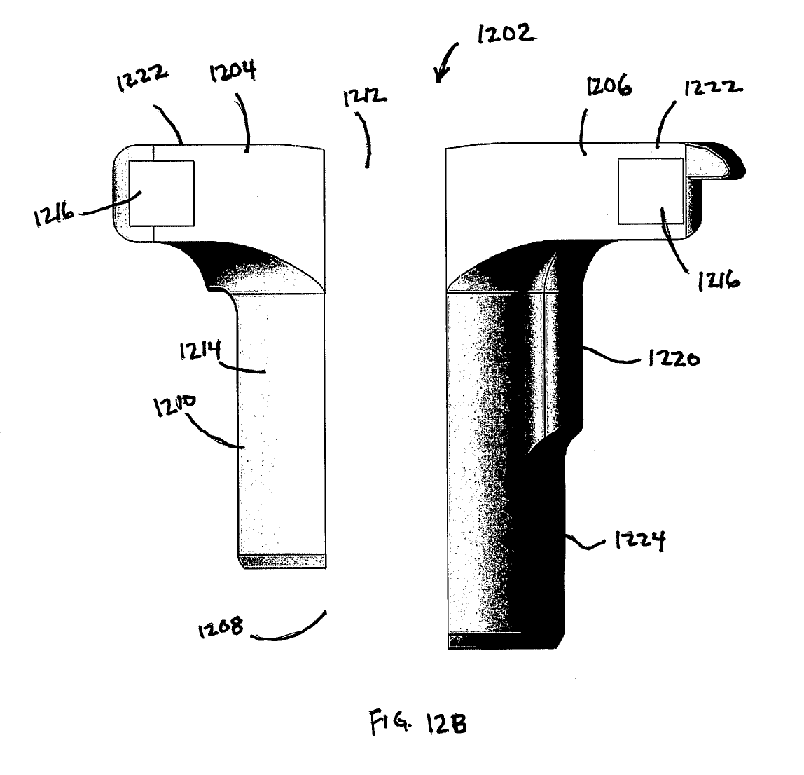

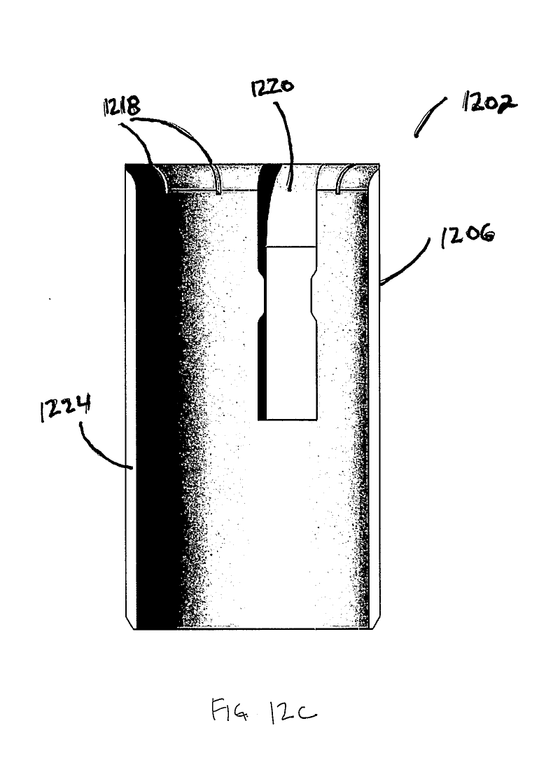

In yet other embodiments, the access device includes a second window opposite the first aperture to allow insertion of surgical instruments from another angle rather than simply by inserting in the channel. FIG. 13A is similar to previous single piece access embodiments. The access device 1302 has a flange 1304, a longer wall on one section 1306 and a portion of the wall removed to form an aperture having a narrow proximal portion 1308 and a wider distal portion 1310. This embodiment also includes a window 1312 through a wall in the longer wall portion 1306 of the access device. The window 1312 allows surgical instruments to be inserted through the window 1312 and either out the distal end of the access device or through the aperture 1310, 1312. The window allows the surgical instrument to be inserted into the access device at a greater angle relative to the longitudinal axis of the access device as compared to a surgical instrument that is inserted in the proximal end of the device. FIG. 13B illustrates a slight variation of the embodiment in FIG. 13A. The access device 1302 is substantially the same as previously described with the major difference being that the second window 1312 has been moved back proximally toward the proximal edge of the access device so that the window is only enclosed on three sides. The fourth side is open. This further allows a surgical instrument to be inserted into the access device as a greater angle, and more easily from the proximal-most end of the access device. This feature, along with any of the other features described herein may be substituted or combined with other features of the access devices described in this specification.

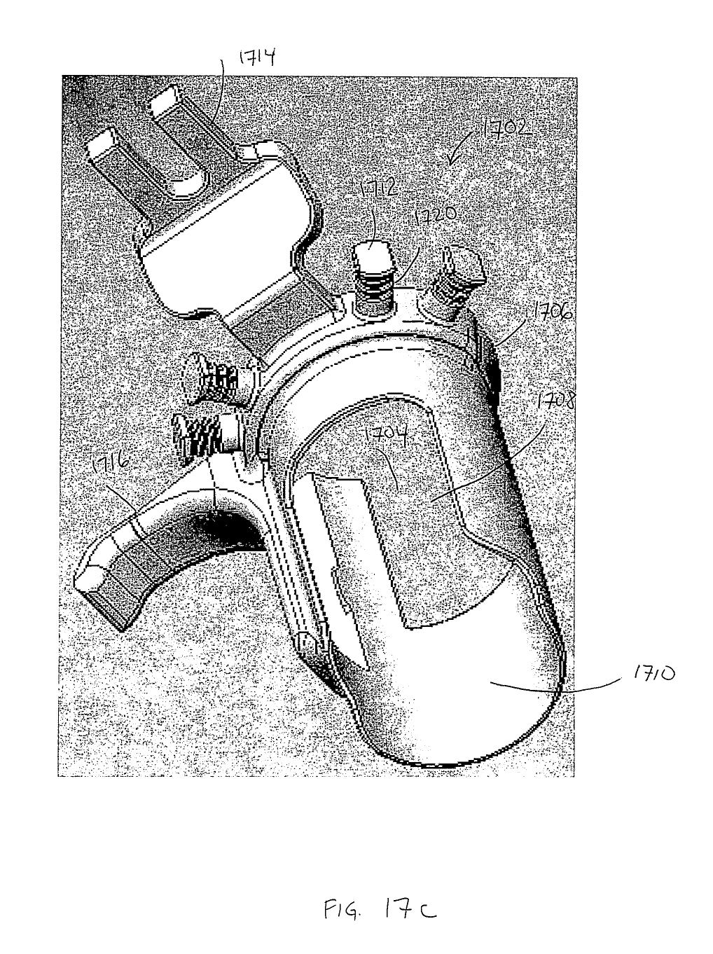

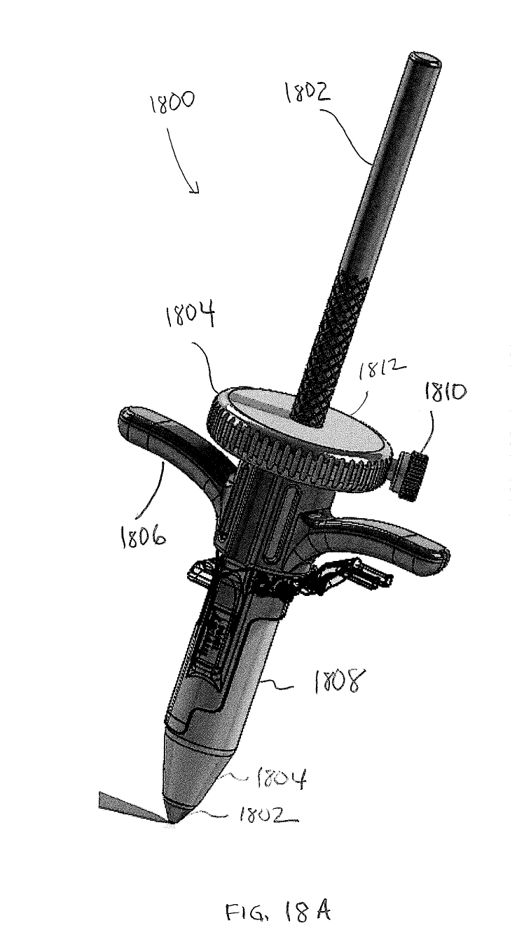

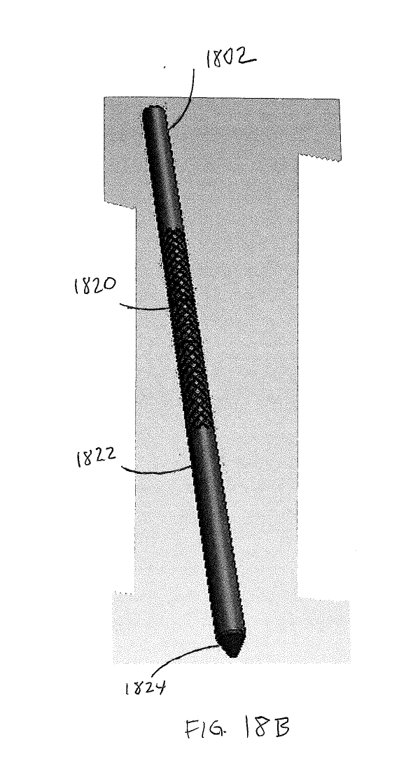

FIGS. 17A-17C illustrate still another exemplary embodiment of an access device. The access device 1702 is similar to other embodiments, except is combines several features from different embodiments previously discussed. FIG. 17A shows a perspective view of access device 1702 which includes two windows 1704, 1708, a flange 1706, suture management features 1712, a coupling element 1714, and an illuminator attachment element 1716. The first window 1708 generally takes the same form as window 1308, 1310 in FIG. 13A or previous embodiments where the window 1708 has a proximal portion and a distal portion. The proximal portion is rectangularly shaped as well as the distal portion 1710. The proximal portion of the window is preferably a long rectangular shape while the distal portion of the window is a narrower and shorter rectangular shape. This embodiment also has a second window 1704 on an opposite side of the access device that is preferably rectangularly shaped. The second window is preferably offset from the first window by approximately 180 degrees so that the first window is on the access side of the device and the second window is on the support side of the device. The access device is also preferably a cylindrically shaped tube but it may have other shapes as well including oval, elliptical, square, rectangular, etc. The suture management feature 1712 and the coupling element 1714 generally take the same form as previously described in other embodiments. The second window allows a surgical instrument to be inserted into the second window instead of the proximal end of the channel, which allows a user to obtain a greater angle with the surgical instrument which facilitates manipulation of the surgical instrument during the surgical procedure. For example, the surgical instrument may be used to push tissue back proximally with a greater degree of angulation, or the instrument can access other regions of the surgical field due to the greater degree of angulation.

FIG. 17B illustrates a perspective view of the access device 1702 taken from above and highlights the top of the device, including the second window 1704, and the illuminator element 1716 which has a recessed region 1718 for receiving an illumination element such as any of the waveguides described herein. The second window in this embodiment extends from the proximal end of the access device distally, but not all the way to the distal end. This view also more clearly illustrates the suture management features 1712 which have circumferential grooves for receiving and holding suture. Other aspects of this embodiment generally take the same form as previously described in other embodiments.

FIG. 17C is yet another perspective view of the access device 1702 taken from another angle, this time emphasizing the first window which has the narrow proximal portion and the wider distal portion, as previously discussed.

The access device of FIGS. 17A-17C may be combined with any of the other devices described in this specification including but not limited to the dilators and illumination elements.

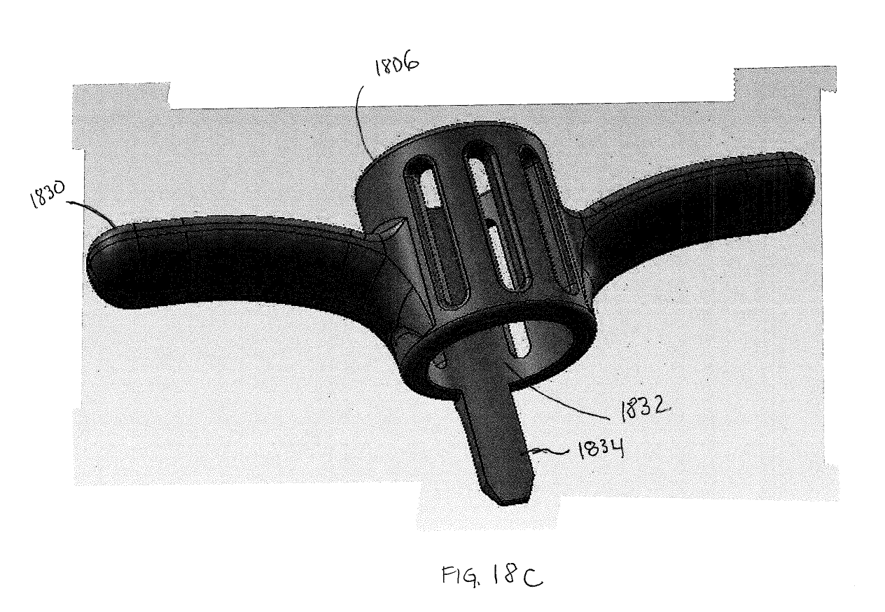

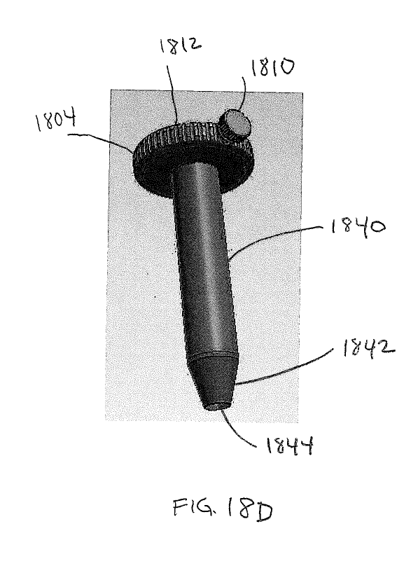

FIG. 18A illustrates a preferred embodiment of an access system that uses the access device of FIGS. 17A-17C. The access system 1800 includes a dilator 1802, an obturator 1804, a grasping element 1806 such as a handle 1806, and an access device 1808. The grasping element 1806 is releasably coupled to both the obturator 1804 and the access device 1808 and the entire assembly of the obturator 1804, grasping element 1806 and access device 1808 is slidably disposed over the dilator 1802. A set screw 1810 on an enlarged flanged portion 1812 of the obturator 1804 may be used as a locking mechanism, and is used to reliably couple the obturator 1804 to the dilator 1802. Other locking mechanisms besides a set screw may be used. For example a spring loaded plunger on the obturator may engage a recessed region on the dilator (or vice versa), or other detent mechanisms, etc. may be used as a locking mechanism to lock the obturator 1804 with the dilator. Alternative embodiments may have a locking mechanism on the grasping element that releasably lock with the obturator while allowing slidably movement of the parts relative to one another.

FIG. 18B illustrates the dilator 1802 in greater detail. The dilator 1802 includes an elongate shaft 1822 having a proximal portion, a distal portion, and a central portion therebetween. The central portion may have surface texturing such as knurling 1820 to allow and operator to firmly grasp the elongate shaft 1822. The distal portion may have a tapered conical or frustoconical tip 1824 to facilitate entry into and through a skin incision and to help dilate tissue as the tip passes therethrough. Preferably, the elongate shaft 1822 is long enough to pass entirely through the obturator 1804, the grasping element 1806, and the access device 1808 such that a portion of the elongate tube extends uncovered proximally beyond the obturator, and also distally beyond the obturator and access device.

FIG. 18C illustrates the grasping element 1806 is greater detail. In this embodiment, the grasping element 1806 is a handle that includes a cylindrical body having a central channel 1832 disposed therethrough for receiving the obturator 1804 and the dilator 1802. A pair of wings 1830 extend radially outward from the cylindrical body and serve as a hand holds so that an operator can easily grasp and manipulate the handle 1806. An optional tab 1834 also extends axially outward from the cylindrical body and serves as a coupling element to engage the handle 1806 with the access device 1808. The access device 1808 may be any of the access devices disclosed in this specification, but preferably may be the access device seen in FIGS. 17A-17C which has a recessed region for receiving an optical element such as a waveguide. The tab 1834 may be sized to fit snugly in the recessed region to help the handle 1806 securely but releasably engage the access device 1808. Other grasping elements may be used instead of a handle with wings. For example, the grasping element may have a knurled cylindrical region that can be used by an operator to hold the device, or recessed regions on the grasping element may also be used to help an operator hold the device. Other grasping elements are well known in the art.

FIG. 18D illustrates the obturator 1804 more closely. The obturator 1804 includes an elongate shaft 1840. A flanged portion 1812 is near the proximal portion of the elongate shaft 1840. The flanged portion is preferably a cylindrical region which surface texturing such as knurling to facilitate grasping and manipulation by an operator. A set screw 1810 is threadably engaged with the flanged region and allows the obturator to be secured to the dilator 1802. Other locking mechanism besides a set screw have previously been described above. A distal portion of the obturator is tapered or conically or frustoconically shaped 1842 to facilitate loading into the access device 1808. The taper also helps insertion of the obturator through the incision and through the tissue as it is advanced into the surgical field. A central channel 1844 is also sized to receive the dilator 1802. The obturator has a length that is long enough to extend past the distal end of the access device 1808. The flanged portion 1812 also has large enough diameter to allow the grasping element 1806 to be captured between the flanged portion and the proximal portion of the access device 1808.

In use, the dilator may be inserted into the incision first. The assembly of the obturator, grasping element and access device is then advanced over the dilator. The assembly is then secured to the dilator by tightening set screw 1810. Thus, the tips of the dilator, obturator and access device line up with one another to form a smooth tapered tip that can easily be advanced distally into the tissue in the surgical field. The entire assembly can then be further advanced distally into the incision to dilate the tissue and distract bones. This is accomplished by grasping and manipulating the wings on grasping element 1806. The access device may be further advanced distally into the surgical field, or it may be rotated to line up the access device with the repair targets in the surgical field. Once the desired position is obtained and the tissue is retracted along with the bone distracted, the set screw may be loosened and the obturator, grasping element and dilator removed. The access device is left in place and the surgical repair procedure may proceed. This procedure is similar to other methods described herein, with the major difference being that a single dilator is used, and the assembly is releasably coupled together during insertion.

While the access device embodiments described above have generally been straight tubes where the walls of the tube are substantially parallel with one another, one of skill in the art will appreciate that this is not intended to be limiting and other designs may also be used. For example, in some situations it may be desirable to use a tapered tube for the access device so that the walls of the tube converge toward one another and thus the distal end has a smaller size than the proximal end. This may be useful for providing maximum access at the proximal end while allowing the distal end to access smaller or confined areas of the body.

Illuminator. As previously mentioned above, it is often desirable to couple an illuminator with the access device to provide illumination to the surgical repair site. LED lights, traditional light bulbs or fiber optics may serve as the illuminator and they may be coupled with the access device, but these do not always deliver light efficiently, or provide the desired quality of lighting needed for viewing a surgical field. Fiber optics are promising but tight loss results in localized heating which can cause burns or even fires. Additionally, the light delivered by fiber optics may not be of suitable quality to enhance visualization of the surgical repair site. Optical waveguides coupled with the access device allow light to be delivered more efficiently and with more desirable properties thereby providing better quality illumination to the surgical field. Therefore, in preferred embodiments, a non-fiber optic optical waveguide is coupled with the access device. In still other preferred embodiments, all or a portion of the access device may be fabricated from an optical material such as acrylic, polycarbonate, silicone, cyclo olefin polymer or cylco olefin copolymer so that the access device is the waveguide. This may be accomplished by injection molding or other processes known in the art. Thus, the waveguide is preferably a single homogenous material.

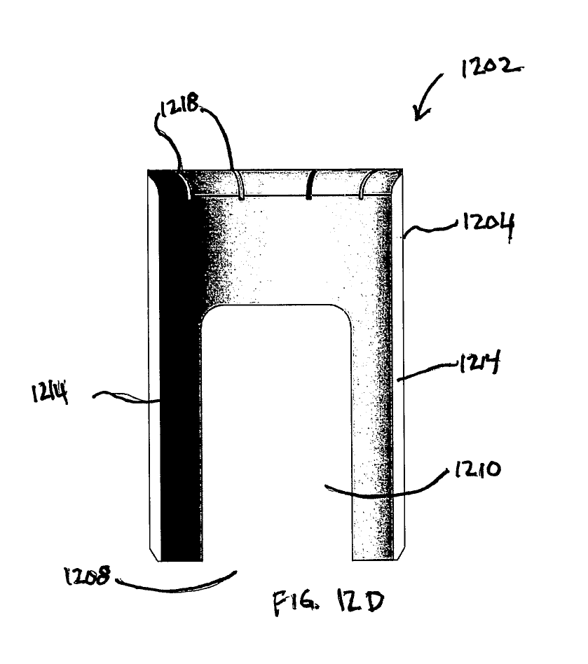

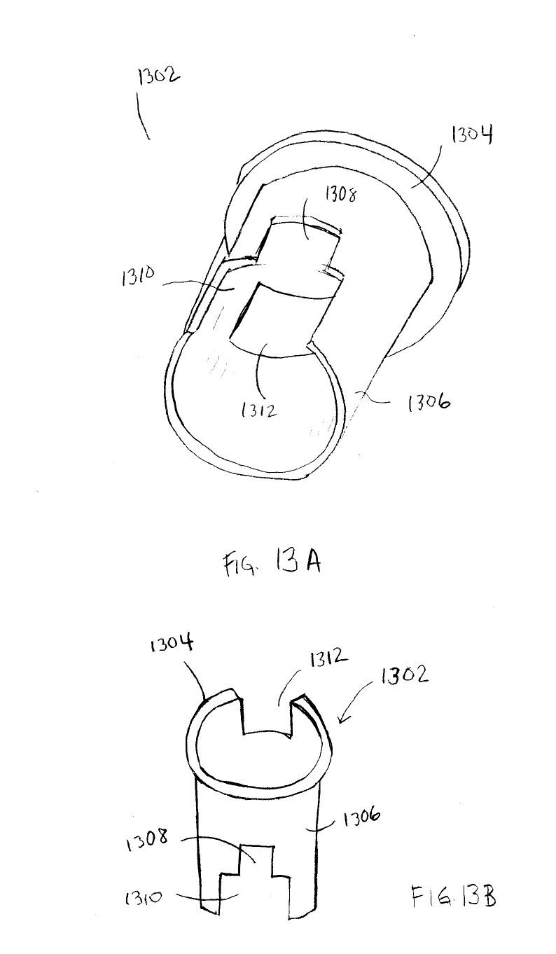

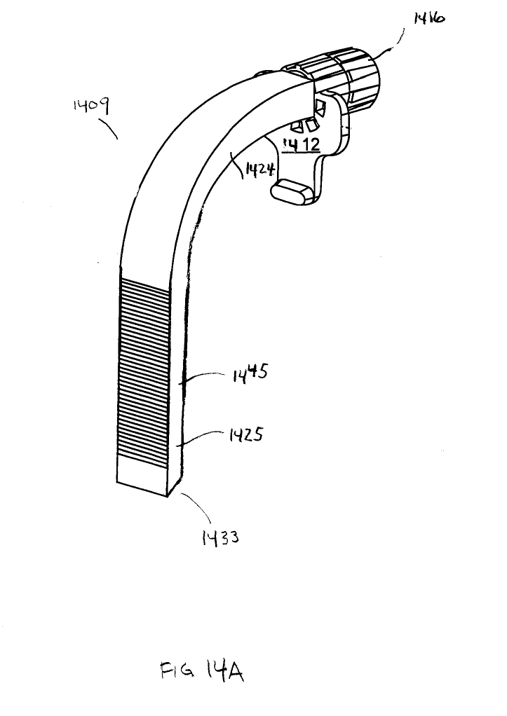

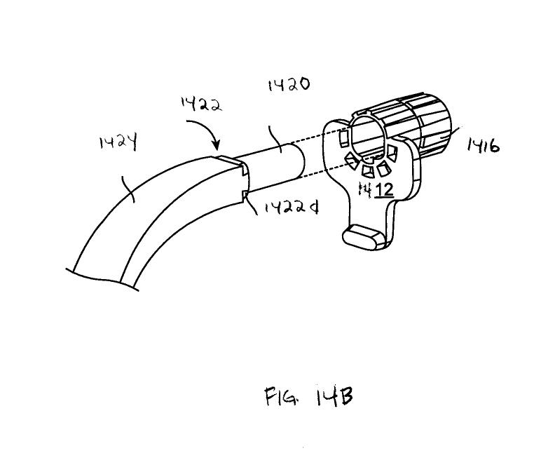

FIGS. 14A-14E illustrate an exemplary illuminator that may be used with any of the access devices described herein. The illuminator is preferably a non-fiber optic optical waveguide formed by injection molding a polymer such as cyclo olefin copolymer or cyclo olefin polymer into a single homogeneous structure that can be coupled with the access device.

FIG. 14A is a perspective view of a waveguide 1409 which may be coupled with the access devices described above. FIG. 14B is an exploded view of the input collar and the waveguide in FIG. 14A. FIG. 14C is a cross-sectional view of the waveguide coupled to an access device. FIGS. 14D and 14E are side and front views of the waveguide, respectively.

The waveguide 1409 may be snap fit into a channel in the access device so that the waveguide is releasably coupled to the access device 1408. Additionally, the channel in the access device allows the waveguide to maintain a low profile when inserted in the access device so as to not take up valuable space. The waveguide optionally may include an engagement element such as plate 1412 to help secure the waveguide to the access device by engagement with an engagement receptacle or recess on the access device. Plate 1412 is joined to collar 1416, and when collar 1416 removably engages input dead zone 1422D (best seen in FIG. 14B), the collar surrounds illumination blade input 1418 as seen in FIG. 14C. There is little or no light transmitted by total internal reflection in a dead zone, thus contact in a dead zone of the waveguide results in minimal or no light loss at that point. The removable engagement of collar 1416 to input dead zone 1422D also brings plate 1412 into contact with an end surface of the access device. Collar 1416 securely engages dead zone 1422D and surrounds cylindrical input zone 1420 and forms input air gap 1420G that is circumferentially disposed around the input zone 1420 thereby minimizing light loss. Engagement at dead zones minimizes interference with the light path by engagement elements such a plate 1412. Optional plate 1412 engages an end surface of the access device and the waveguide either snap fits or is snugly engaged with the channel of the illuminator attachment element on the access device to hold the waveguide 1409 fixed to the access device without contact between active zones of waveguide 1409 and any part of the access device. This minimizes light loss from the waveguide due to contact.

Waveguide 1409 is configured to form a series of active zones to control and conduct light from input 1418 of the cylindrical input zone 1420 to one or more output zones such as output zones 1427 through 1431 and output end 1433 as illustrated in FIGS. 14D-14E. Waveguide 1409 also includes one or more dead zones such as zones 1422D. Dead zones are oriented to minimize light entering the dead zone and thus potentially exiting in an unintended direction. As there is minimal light or no light in or transiting dead zones by total internal reflection they are ideal locations for engagement elements to secure the illumination blade to the retractor.

Light is delivered to input 1418 using any conventional mechanism such as a standard ACMI connector preferably having a 0.5 mm gap between the end of the fiber bundle and input 1418, which is preferably 4.2 mm diameter to gather the light preferably from a 3.5 mm fiber bundle with a preferably 0.5 NA. Light incident to input 1418 enters the waveguide through generally cylindrical, active input zone 1420 and travels through active input transition 1422 to a generally rectangular active neck 1424 and through output transition 1426 to output region 1425 which contains active output zones 1427 through 1431 and active output end 1433. Neck 1424 is generally rectangular and is generally square near input transition 1422 and the neck configuration varies to a rectangular cross section near output transition 1426. Output region 1425 has a generally high aspect ratio rectangular cross-section resulting in a generally wide and thin blade. Each zone is arranged to have an output surface area larger than the input surface area, thereby reducing the temperature per unit output area.

In the illustrated configuration waveguide 1409 includes at least one dead zone, dead zone 1422D, generally surrounding input transition 1422. One or more dead zones at or near the output of the waveguide provide locations for engagement elements such as tabs or standoffs to permit stable engagement of the waveguide to the access device. This stable engagement supports the maintenance of an air gap such as air gap 1421 adjacent to all active zones of the waveguide as illustrated in FIG. 14C. Neck zone 1424 ends with dimension 1432 adjacent to output transition 1426 which may extend to dimension 1434 at the output zones, or the width may remain constant along the length of the waveguide.

Output zones 1427, 1428, 1429, 1430 and 1431 have similar configurations with different dimensions. Referring to the detailed view of FIG. 14D, the characteristics of output zone 1427 are illustrated. Each output zone is formed of parallel prism shapes with a primary surface or facet such a primary facet 1440 with a length 1440L and a secondary surface or facet such as secondary facet 1442 having a length 1442L. The facets are oriented relative to plane 1443 which is parallel to and maintained at a thickness or depth 1444 from rear surface 1445. In the illustrated configuration, all output zones have the same depth 1444 from the rear surface.

The primary facets of each output zone are formed at a primary angle 1446 from plane 1443. Secondary facets such as facet 1442 form a secondary angle 1447 relative to primary facets such as primary facet 1440. In the illustrated configuration, output zone 1427 has primary facet 1440 with a length 1440L of 0.45 mm at primary angle of 27 degrees and secondary facet 1442 with a length 1442L of 0.23 mm at secondary angle 88 degrees. Output zone 1428 has primary facet 1440 with a length 1440L of 0.55 mm at primary angle of 26 degrees and secondary facet 1442 with a length 1442L of 0.24 mm at secondary angle 66 degrees. Output zone 1429 has primary facet 1440 with a length 1440L of 0.53 mm at primary angle of 20 degrees and secondary facet 1442 with a length 1442L of 0.18 mm at secondary angle 72 degrees. Output zone 1430 has primary facet 1440 with a length 1440L of 0.55 mm at primary angle of 26 degrees and secondary facet 1442 with a length 1442L of 0.24 mm at secondary angle 66 degrees. Output zone 1431 has primary facet 1440 with a length 1440L of 0.54 mm at primary angle of 27 degrees and secondary facet 1442 with a length 1442L of 0.24 mm at secondary angle 68 degrees. Thus, the primary facet 1440 in preferred embodiments forms an acute angle relative to the plane in which the rear surface 1445 lies, and the secondary facet 1442 in preferred embodiments forms an obtuse angle relative to the plane in which the rear surface 1445 lies. These preferred angles allow light to be extracted from the waveguide so that light exits laterally and distally toward the surgical field in an efficient manner, and the waveguide to be injection molded and easily ejected from the mold. Other angles are possible, as will be appreciated by one of skill in the art.

Output end 1433 is the final active zone in the waveguide and is illustrated in detail in FIG. 14D. Rear reflector 1448 forms angle 1449 relative to front surface 1450. Front surface 1450 is parallel to rear surface 1445. Terminal facet 1451 forms angle 1452 relative to front surface 1450. In the illustrated configuration, angle 1449 is preferably 32 degrees and angle 1452 is preferably 95 degrees. This distal tip geometry helps to prevent light from reflecting back proximally toward the physician, thereby helping to prevent glare.

Other suitable configurations of output structures may be adopted in one or more output zones. For example, output zones 1427 and 1428 might adopt a concave curve down and output zone 1429 might remain generally horizontal and output zones 1430 and 1431 might adopt a concave curve up. Alternatively, the plane at the inside of the output structures, plane 1443 might be a spherical section with a large radius of curvature. Plane 1443 may also adopt sinusoidal or other complex geometries. The geometries may be applied in both the horizontal and the vertical direction to form compound surfaces.

In other configurations, output zones may provide illumination at two or more levels throughout a surgical site. For example, output zones 1427 and 1428 might cooperate to illuminate a first surgical area and output zones 1429 and 1430 may cooperatively illuminate a second surgical area and output zone 1431 and output end 1433 may illuminate a third surgical area. This configuration eliminates the need to reorient the illumination elements during a surgical procedure.

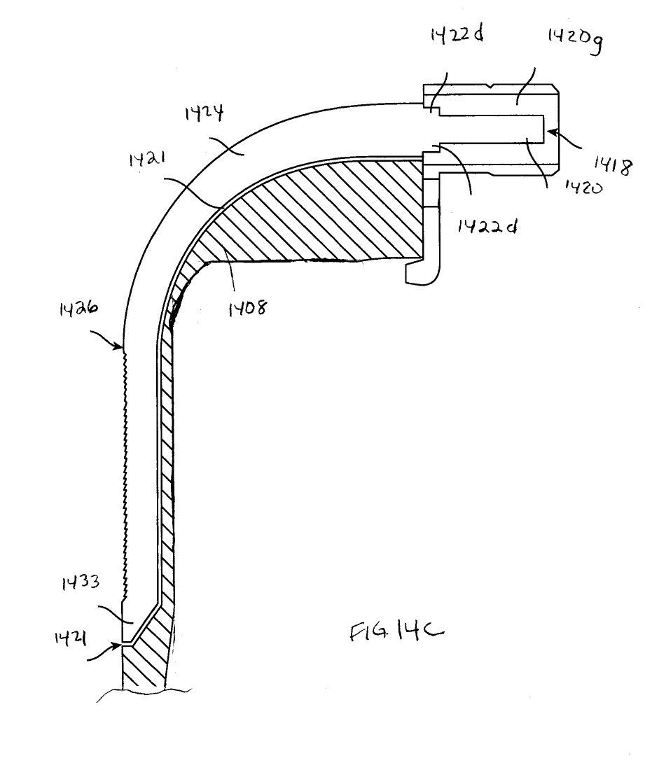

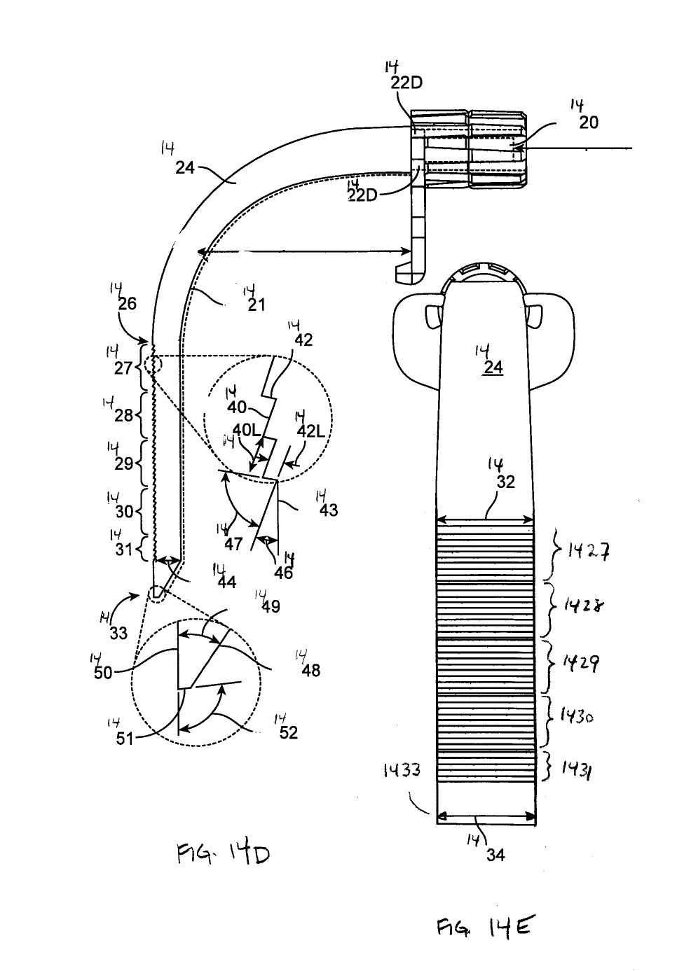

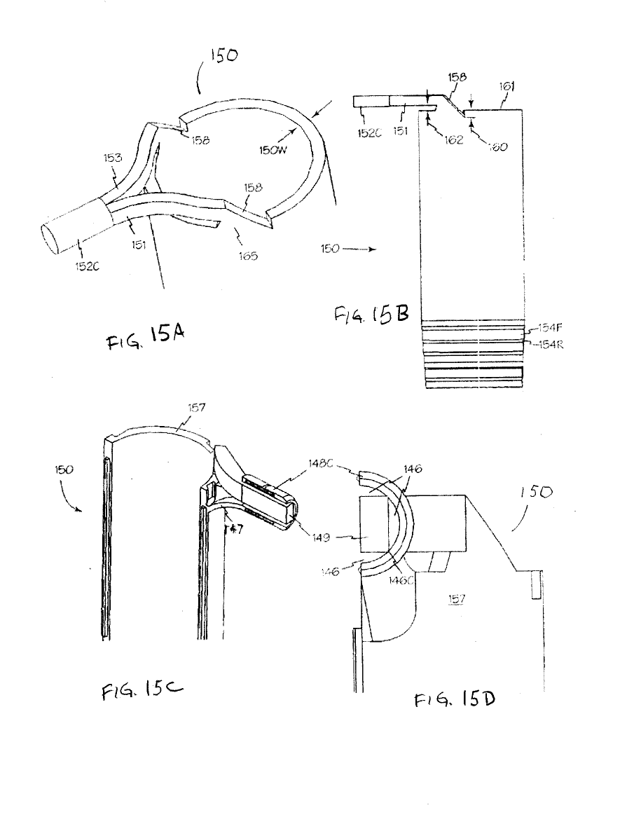

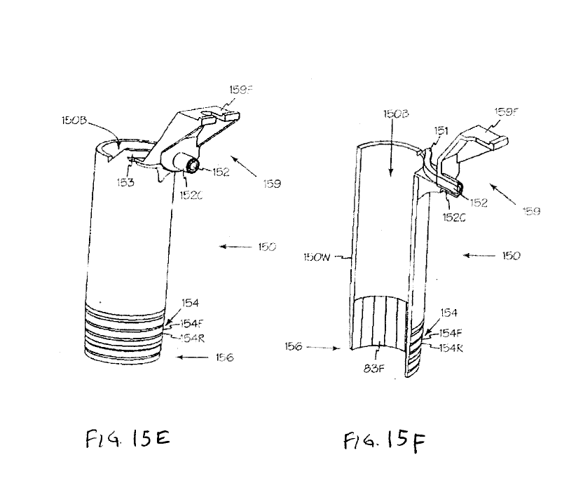

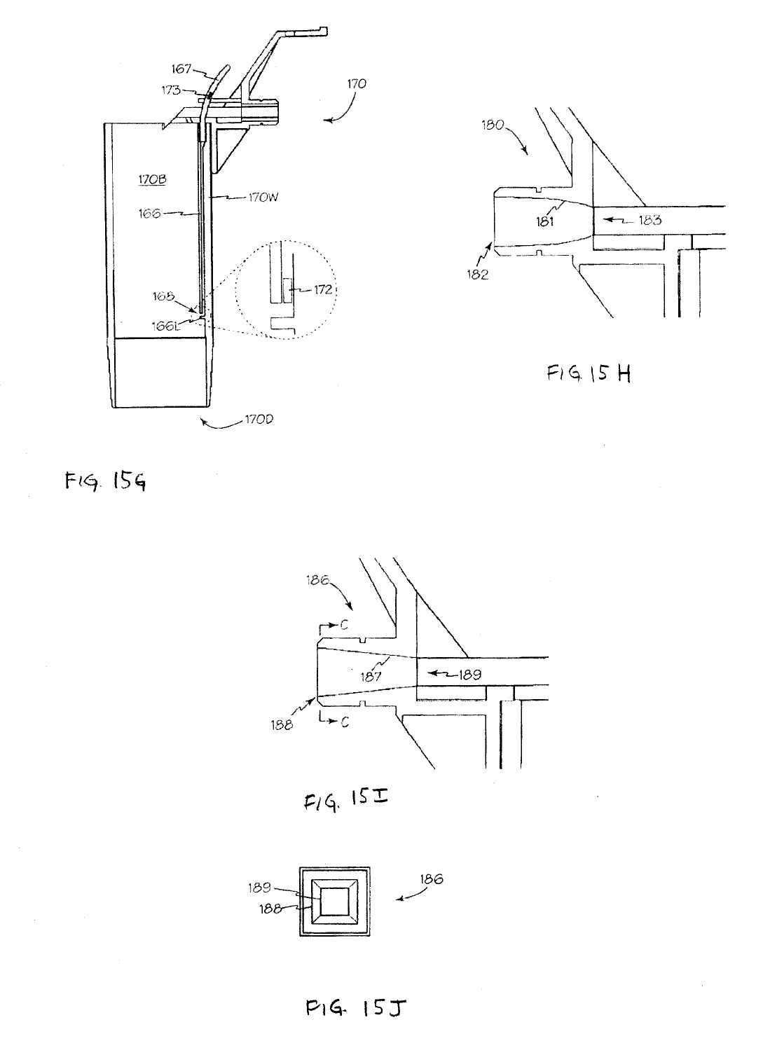

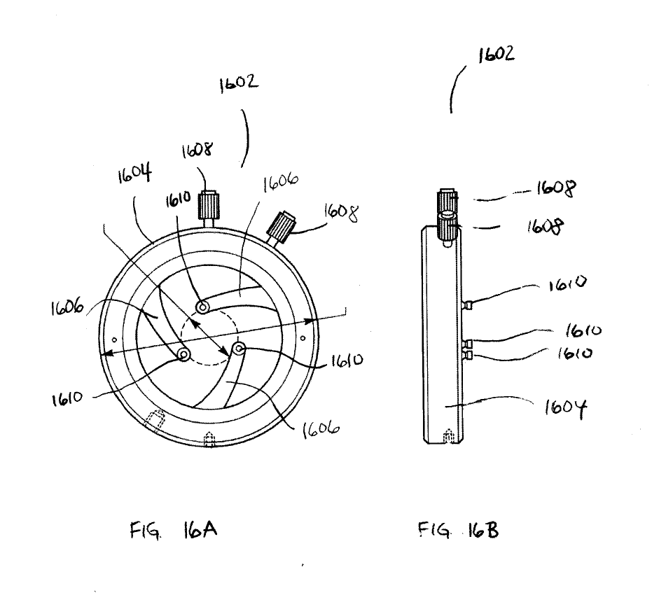

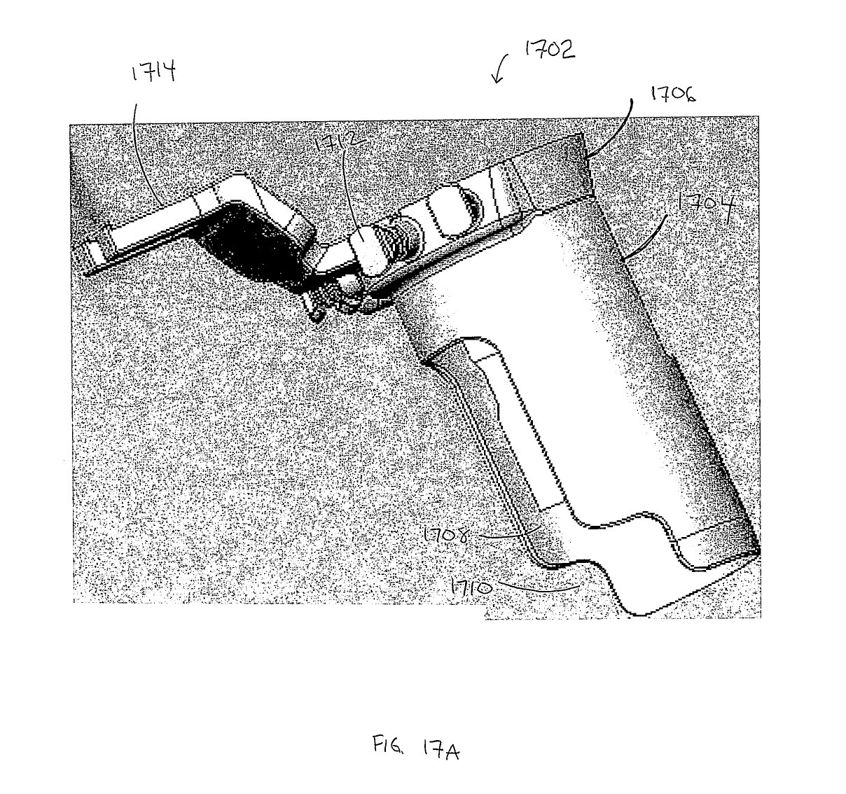

In order to provide circumferential illumination of the surgical field, a tubular waveguide may be inserted into the channel of the access device. Exemplary embodiments of such a tubular waveguide and various features which may be used in any of the illuminators disclosed herein are illustrated in FIGS. 15A-15J.

FIG. 15A illustrates a perspective view of the top of optical waveguide 150 and FIG. 15B is a side view. The waveguide is formed into a tube or cannula and is illustrated without clamp flange/holder 159 in place (best seen in FIG. 15E). Input arms 151 and 153 are offset above proximal surface 161 by a distance 162 and end in angled reflector surface 158 that partially extends down distance 160 into the tube wall. The offset controls the light entering waveguide 150 and restricts light entering into input structure 165. The input arms may be separate components attached to the waveguide or they may be integrally formed with the waveguide (e.g. by injection molding). In alternative embodiments, a short flexible fiber optic pigtail may be coupled to the waveguide either by potting the fiber optic pigtail with the waveguide or by molding the waveguide around the pigtail. Reflector surface 158 serves to direct light orthogonally from the horizontal input and down into the tube wall, also causing the light to spread around the circumference of the tube wall by the time the light reaches the distal or lower part of the tube. Reflector surfaces such as surface 158 may be a flat surface, an arced surface, or a series of interconnected surfaces and may also end at the top of the tube wall. Reflector surface 158 may be treated, e.g., a reflective or metalized coating or an applied reflective film, to enhance reflection.

Air gaps may be used to isolate the light-conducting pathway in any suitable connector. Waveguide 150 in FIG. 15C includes male connector 148C that has been integrated with waveguide tube wall 157 via bracket 147. This allows connector 148C to be molded with the waveguide and not attached as a separate part, such as a standard light connector. A separate connector introduces tolerance concerns into the system that may result in reduced coupling efficiency between a fiber optic cable output and waveguide input 149 because the two parts may not be aligned correctly. Molding the connector and the waveguide input as one piece substantially reduces the chance of misalignment and thereby increases coupling efficiency.

FIG. 15D is a front view looking into the input of connector 148C. Air gaps 146 are maintained around waveguide input 149 to isolate the light-conducting pathway. One or more small zones of contact such as contact zone 146C may be maintained, essentially bridging connector 148C and input 149 with a small amount of material, to add strength and stability to the system while resulting in minimum light loss in the contact zone.

Referring to FIGS. 15E-15F light input connector 152C surrounds light input cylinder 152 which may be divided into multiple input arms such as arms 151 and 153 that then direct light into illumination waveguide 150. Input arms 151 and 153 may assume any suitable shape and cross-sections depending on the optical design goals, such as the multi-radius arms with rectangular cross-section shown or straight sections (no radius) or angle rotators, etc. Also shown is a clamp flange holder 159 that serves to support input connector 152C and arms as well as providing a standard light connector 152C over input cylinder 152 (e.g., an ACMI or WOLF connector) and a flange 159F at the top for attaching a clamp used to hold the entire structure in place once it is positioned relative to a surgical site in a body. A shelf or other similar light blocking structures may be added to the holder, extending over the input arms and/or the upper tube edge as needed to help block any light that may escape these structures that might shine up into the user's eyes. Optional circumferential light extraction structures 154 are shown at the bottom, distal end 156, of the tube. In the section view of FIG. 15F, optional vertical light disruption structures or facets 83F are shown on the inside wall of the tube.

A clamp adapter 159F that also support light coupling 152C for introducing light energy into cannula 150. The relative orientation of the clamp adapter and the light coupling as shown enables the clamp adapter to operate as a shield to prevent any misdirected light shining into the eyes of anyone looking into bore 150B of the cannula, but the clamp adapter and light coupling may adopt any suitable orientation.

FIG. 15F illustrates optional vertical facets 83F within the distal end for disrupting the light spiraling within the waveguide. Circumferential light extraction structures 154 may include stepped facets such as facets 154F and risers such as riser 154R on the outside tube wall 150W. The “riser” section of the stepped facet section 154R is angled so that it may slide against tissue without damaging the tissue. Steps may be uniform or non-uniform depending on the light directional control desired. The steps may be designed to directly light substantially inwards and toward the bottom of the tube or some distance from the bottom of the tube, or they may be designed to direct light toward the outside of the tube, or both.

Circumferential light extraction structures such as structures 154 may be facets or may be other geometries, such as parabolas. Circumferential light extraction structures coupled with light directing structures that provide circumferentially distributed light to the extraction structures provide circumferential illumination. Since tools entering the interior of the tube now have light shining on them from all sides, the tools do not cast any shadows within the cone of illumination emitted by the cannula. The circumferential illumination from a cylindrical waveguide creates a generally uniform cone of light that minimizes shadows, e.g., from instruments, creating substantially shadowless illumination in the surgical field below the tubular waveguide.

Referring now to FIG. 15G, optional structure 166 along the inside wall may be used for suction for smoke evacuation and or ventilation. Smoke from an electrosurgical knife may obscure the surgeon's view until the smoke dissipates. A ventilation tube such as tube 167 may be attached to the top of structure 166 to engage the suction structure and provide a source of suction or vacuum. The bottom of suction structure 166 may be as shown opening into working channel 170B orthogonal to wall 170W or it may open directly toward the bottom or distal end 170D by removing lower lip 166L. The former is preferred to reduce the chance that debris is sucked into the suction structure thereby blocking it. One or more additional tubes may also be positioned to inject air into the cannula bore, angled along the walls to create a vortex-like air flow that draws smoke toward the side walls where it can then be evacuated, the air flow serving to clear the smoke sooner from the center of the tube where it may obscure vision. In alternative embodiments, the ventilation tube may be formed integrally in a wall of the waveguide, thereby minimizing profile of the device and minimizing protrusions.

Small filters such as debris filter 172 may be included in or near suction input 168 to block debris. The lower suction opening, input 168, is preferred to be as close to distal end 170D of illuminated waveguide 170 as practical, while not interfering with the optical structures, in order to evacuate smoke from electrocautery as soon as possible. Multiple suction openings may be provided along the vertical channel of the suction section, but these ports should be sized differently, smallest at the top and largest at the bottom so that there is sufficient suction at the bottom port. The suction ports and channel should be designed to minimize turbulence that contributes to noise. Multiple suction structures may be provided. A shelf in clamp flange/holder may help secure the suction tubing to the suction source. Suction tubing 167 or suction structure 166 in tube 170 may also include one or more air filters 173, e.g., charcoal filters, to remove the smell of the smoke and or other airborne impurities.

Referring now to FIG. 15H, input coupling 180 may incorporate compound parabolic concentrator 181 or a similarly functioning device, such as optical taper 187 in FIG. 151, whose input 182 is sized to match the largest fiber bundle, which is typically 5 or 6 mm in diameter, but whose output 183 is coupled to a smaller waveguide thickness, e.g., 3 or 5 mm. Such a device could be hidden in the connector of the waveguide device, e.g., inside of an ACMI connector or other suitable device.

Input 188 of optical taper coupling 186 in FIGS. 15I-15J provides a significant improvement in input coupling and occurs by using a square input coupler on the waveguide that couples to a typical, round fiber bundle cable. The increase in coupling surface area results in improved light coupling for a variety of fiber sizes without the effect on numerical aperture. Also, an index matching material may used between the fiber optic and the input to the waveguide to improve optical coupling. Other aspects of a cylindrically shaped optical waveguide are disclosed in US Patent Publication No. 2009/0036744, the entire contents of which are incorporated herein by reference.

Additional details and features related to optical waveguides which may be used with the waveguides disclosed herein or other waveguides, and which may be combined with the access device are disclosed in the follow US Patents and Publications: US Patent Publication Nos. 2007/0208226; 2012/0083663; 2010/0041955; 2012/0041268; and U.S. Provisional Patent Application No. 61/705,027; and U.S. Pat. Nos. 7,686,492; 8,047,987; 8,088,066; the entire contents of each is incorporated herein by reference.

The vacuum feature as well as any of the other features described herein may be substitutes with other features or combines with other features described herein. Additionally, any of the waveguides may be coated or clad with a film to enhance optical properties. For example, a film may be used to polarize light extracted from the waveguide. Or various coatings or cladding (e.g. heat shrink) may be used minimize light loss from the waveguide by enhancing total internal reflection of the light. In still other embodiments.

Rotator Cuff Repair: The following surgical procedure is an exemplary method of retracting tissue and distracting bone in a rotator cuff repair using the surgical access system of FIGS. 2A-2C or any of the embodiments disclosed herein.

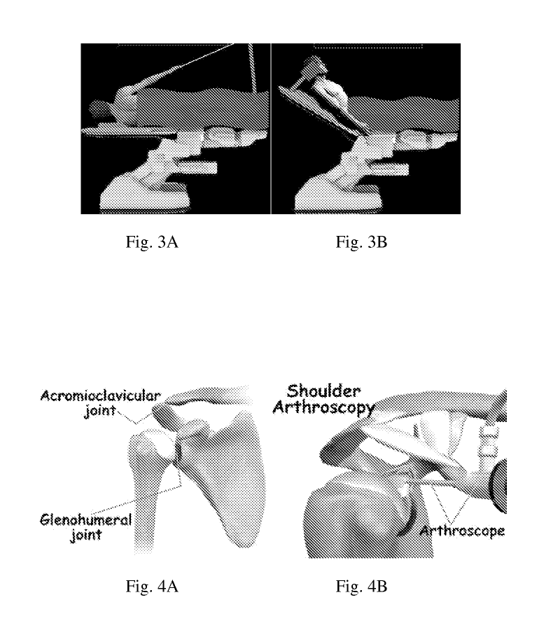

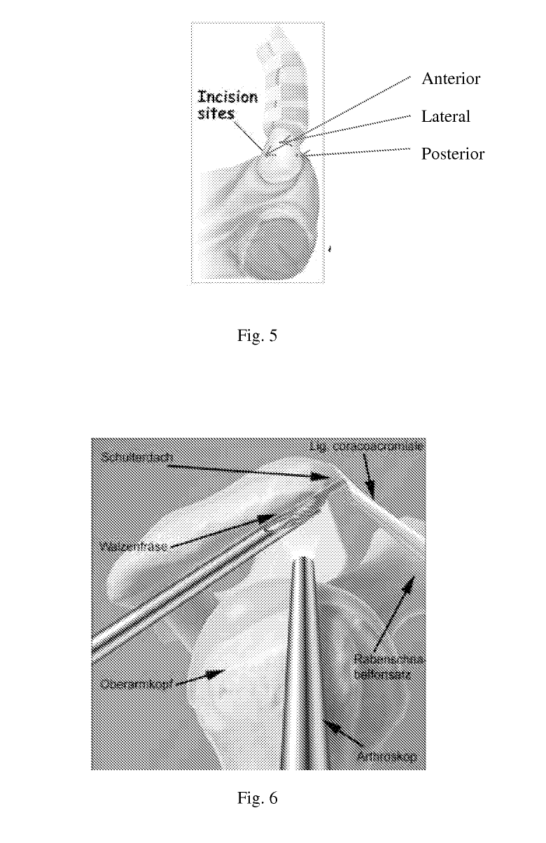

The patient is placed in either a lateral decubitus or modified beach chair type of position on the operating table as illustrated in FIGS. 3A-3B. After the shoulder is prepped and draped in the usual sterile manner, an arthroscopic examination of the shoulder is performed. An arthroscope is inserted into the glenohumeral joint through a posterior portal as seen in FIGS. 4A-4B. Any intra-articular problems, such as a torn labrum, can be addressed at this time. It is at this time that the rotator cuff tear can be analyzed. Utilizing an additional anterior or lateral portal, the size of the rotator cuff tear can be assessed. FIG. 5 illustrates portal locations.

In addition to that, the quality and mobility of the cuff tissue can also be assessed. If the tear is not amenable to repair, a debridement can be performed. If the cuff tear does appear to be amenable to repair, the surgeon can proceed with the following procedure. The arthroscope is then inserted into the subacromial space as seen in FIG. 6.

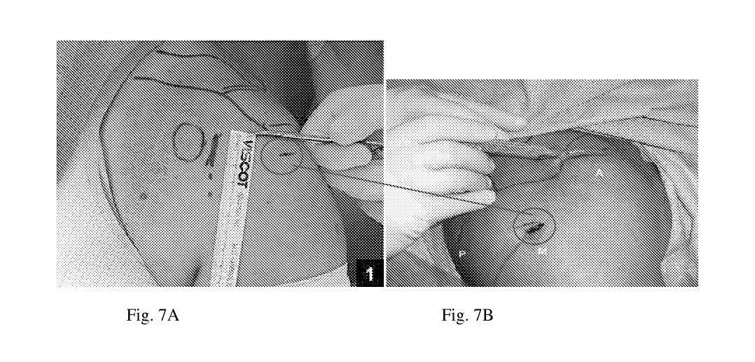

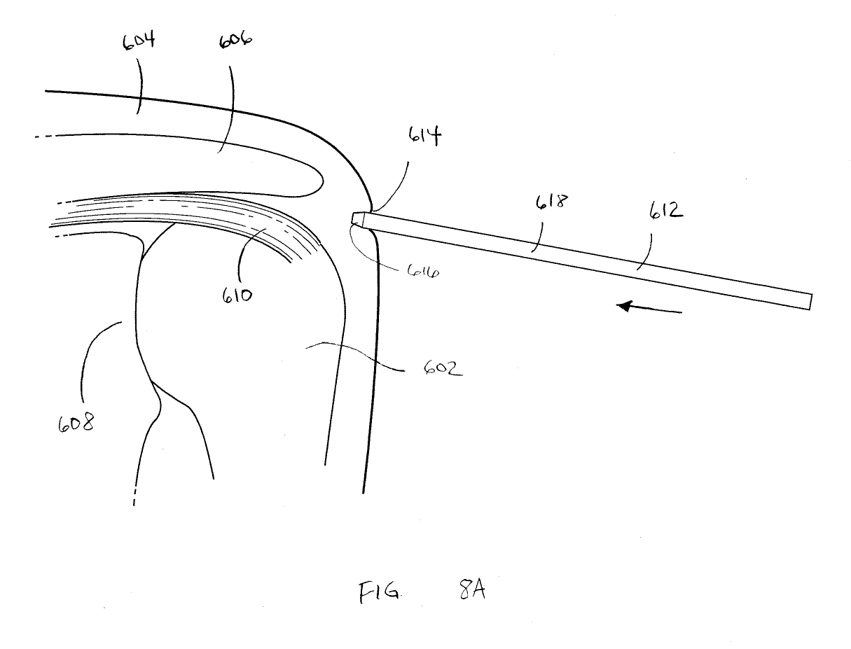

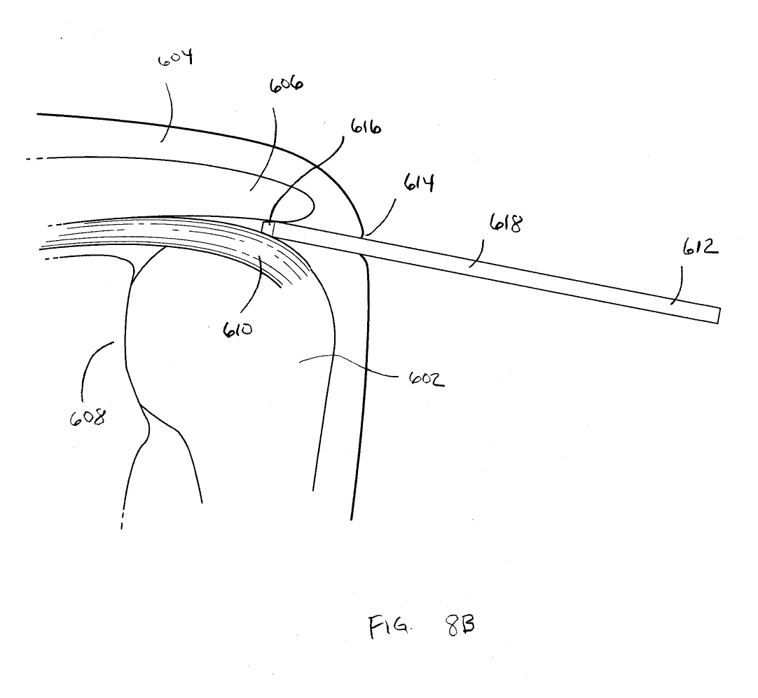

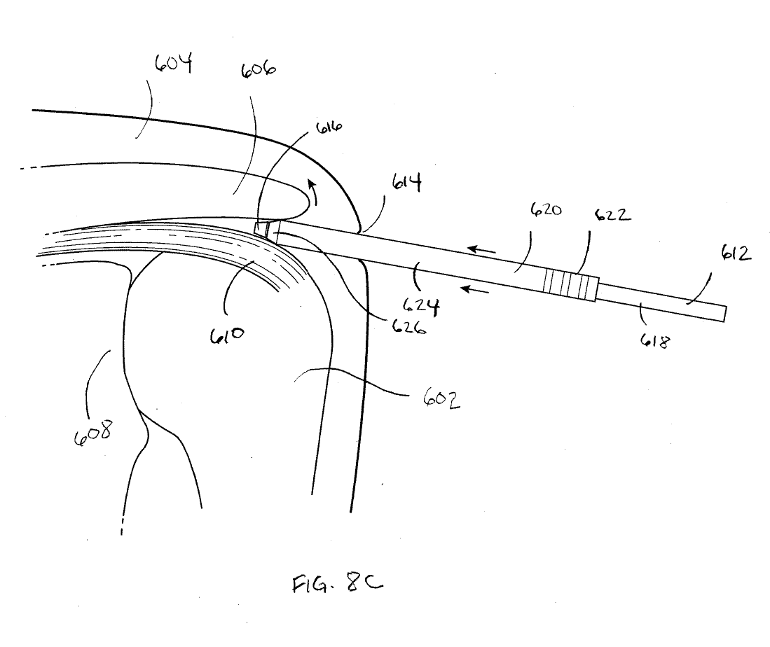

After debridement of any bursal tissue that may be obstructing the view, a spinal needle can be used to establish the location of the lateral portal. Depending on the location of the primary portion of the tear, the portal can be moved either more anterior or posterior relative to the acromion to give the best angle of approach to the tear. Once this position has been determined, a scalpel is used to make a skin incision approximately 3 cm long as illustrated in FIGS. 7A-7B.

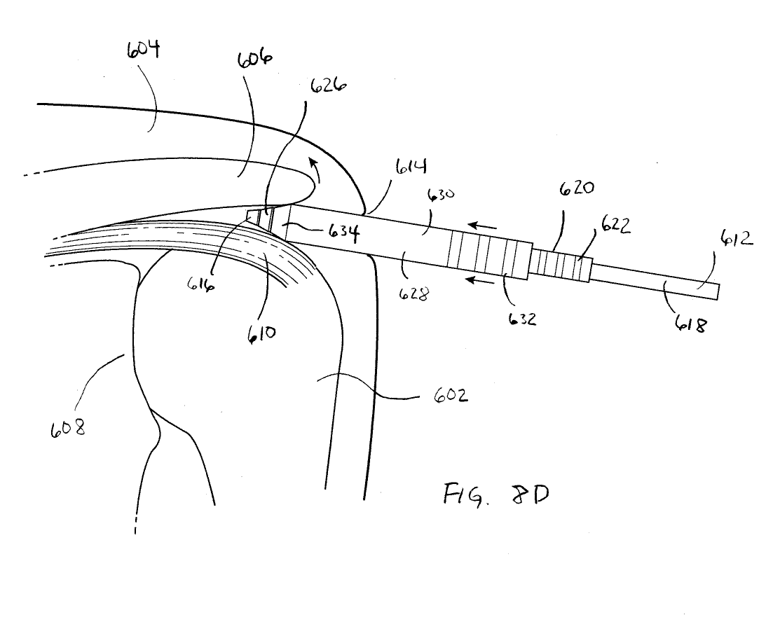

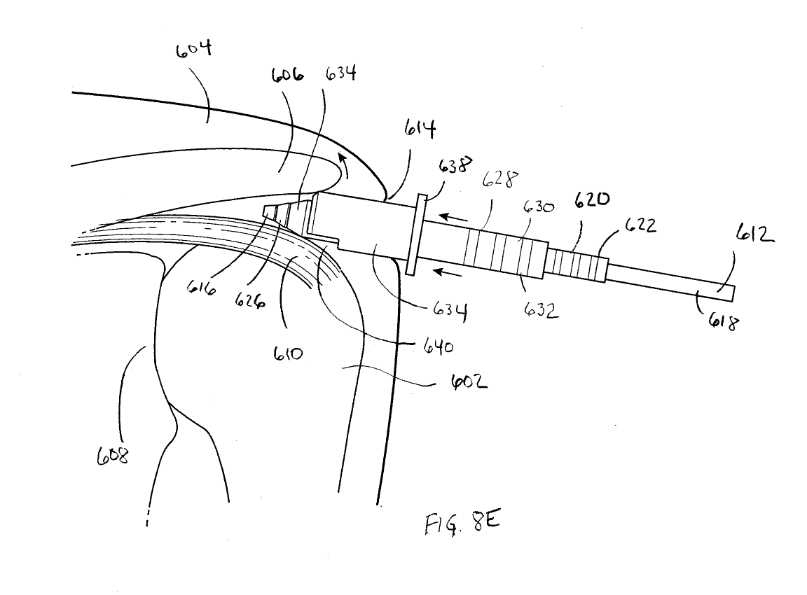

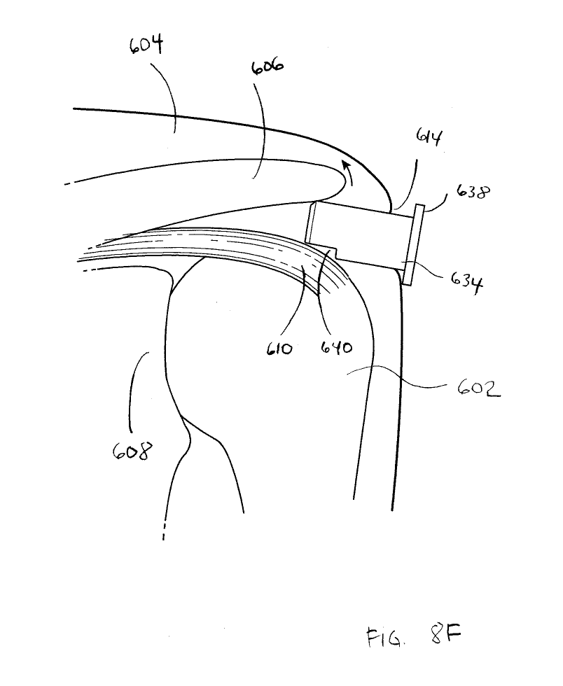

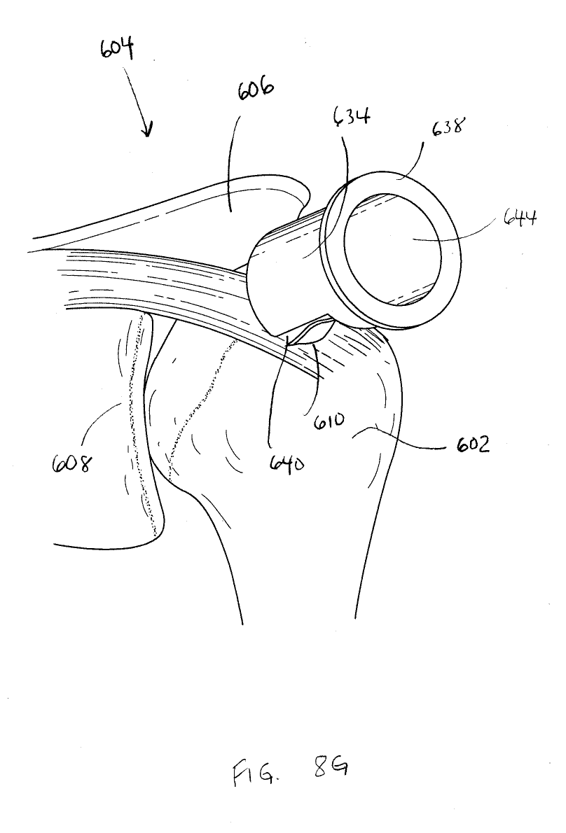

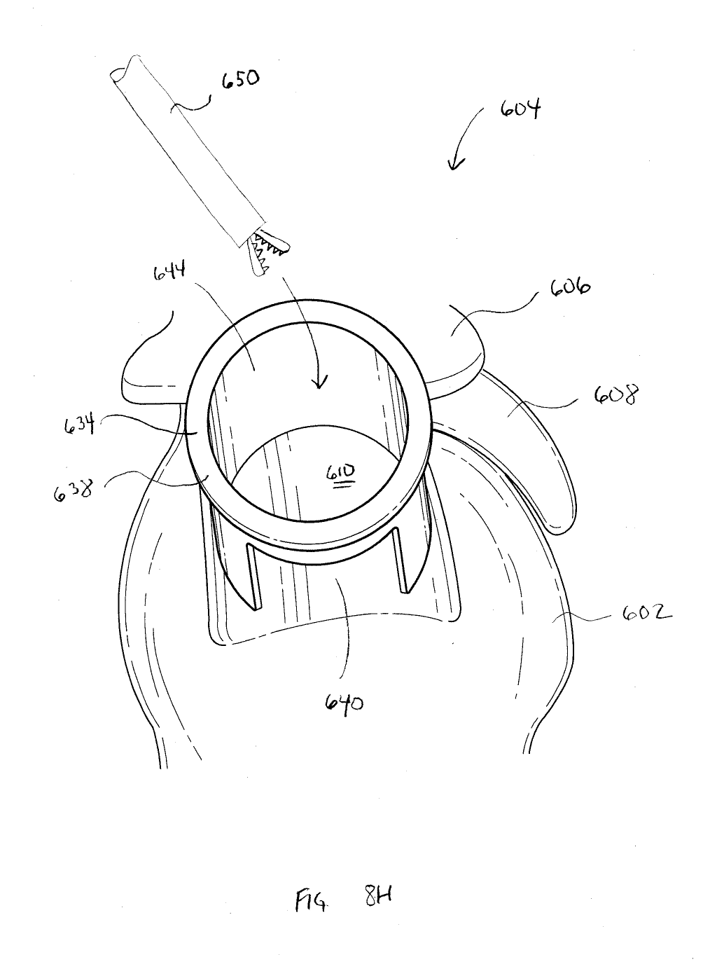

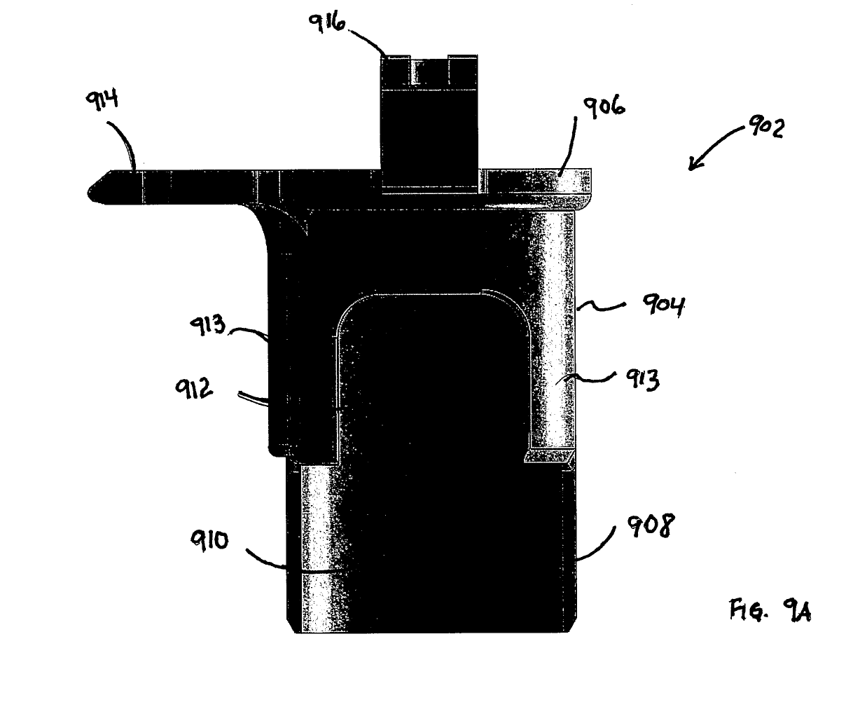

FIGS. 8A-8H illustrate use of any of the access devices disclosed herein in the exemplary rotator cuff repair. FIG. 8A illustrates the shoulder 604 with a humerus 602, glenoid 608, acromion 606 and damaged tendon 610. A dilator 612 is inserted into the incision 614 previously created and advanced distally into the subacromial space in order expand the incision 614 as well as expand adjacent tissue in the shoulder such as the deltoid muscle and distract the acromion away from the humerus. An arthroscope may be used to visualize advancement of the dilators. The dilator preferably includes a tapered or chamfered tip 616 to facilitate its advancement through the tissue and between bones, and also has an elongate shaft 618 for grasping by a surgeon. Using the dilator splits the tissue such as the deltoid muscle along the line of its fibers as opposed to cutting the tissue, thereby minimizing trauma.

In FIG. 8B, the first dilator 612 is advanced further distally until it is disposed between the acromion and the humerus, and also so that the dilator is adjacent the torn tendon 610. The dilator will retract tissue as it is advanced distally, and depending on the size of the dilator, it may begin to distract the acromion away from the humerus.

In FIG. 8C, a second dilator 620 is slidably advanced over the first dilator 612 to expand the incision 614 further. As the second dilator is advanced further distally, it will continue to retract tissue and also distract the acromion and humerus away from one another. The second dilator 620 is similar to the first dilator 612 and has an elongate shaft 624 with a tapered or chamfered tip 626 and also has a textured, knurled or other features on a proximal end to facilitate grasping and manipulation by a surgeon.

In FIG. 8D, a third dilator 628 is advanced over the first and second dilators 612, 620 past the incision thereby expanding the incision 614. As the third dilator 628 is advanced distally, it retracts tissue and further distracts the acromion from the humerus. The third dilator 628 is similar to the first and second dilators 612, 620 and has an elongate shaft 630 with a tapered or chamfered tip 634 and a textured 632, knurled or other features on a proximal end to facilitate grasping and manipulation by a surgeon. The dilators are preferably sized so that their outer diameter is similar to the inner diameter of the dilator that is slidably disposed thereover. The tapered tips are angled so that when all the dilators are inserted in one another and advanced distally, the tip of the assembly has smooth transition. This exemplary embodiment only illustrates the use of three dilators, but any number of dilators may be stacked together and used to retract tissue and distract the bones.

After the desired number of dilators have been inserted into the incision 614 such that the acromion has been distracted from the humerus and soft tissue has been adequately dilated, an access device 634 is then slidably advanced over the largest size dilator through the incision 614 into the subacromial space of the shoulder as seen in FIG. 8E. The access device is advanced distally until it is disposed adjacent the torn tendon 610 to be repaired. The access device simultaneously circumferentially retracts tissue away from the repair site and also distracts the acromion away from the humerus thereby increasing the working space available to perform the surgical repair. The access device has an upper wall portion that is longer than the lower wall of the device. This allows the device to be inserted under the acromion while still providing exposure and access to the area of the rotator cuff footprint on the greater tuberosity. The outer wall of the access device supports tissue and prevents it from collapsing inward while a portion of the wall is removed to create an access aperture 640. This allows a surgical instrument to access the repair site while the remainder of the access device retracts and distracts tissue and bone. The access device may be advanced distally until a flange 638 prevents further advancement. In alternative embodiments, the access device may provide light to illuminate the surgical field. Additional details about the light are described elsewhere in this specification.

Once the access device 634 has been positioned in the shoulder, the dilators 612, 620, 628 may be removed from there as seen in FIG. 8F. The access device creates a clear path for surgical instruments to be inserted into the shoulder to repair the torn tendon 610. The access device also allows visualization of the treatment site since the tissue and bone have been retracted and distracted away. FIG. 8G illustrates the access device 634 inserted into the shoulder 604. In FIG. 8H, a channel 644 allows surgical instruments 650 to be inserted into the shoulder to access the repair site through aperture 640 while the access device retracts tissue and distracts the acromion 606 away from the humerus 602. The surgical method described in FIGS. 8A-8H may be performed with any of the dilators, access devices, and access device

After the access device has been positioned, often with visualization provided by an arthroscope, the arthroscope is then removed. Any remaining bursal tissue in the subacromial space is then removed with a combination of sharp dissection and rongeur. Any adhesions about the cuff can be lysed utilizing a Cobb or Key type elevator. If necessary, the capsular to the cuff can also be lysed with a Cobb or Key elevator. The leading edge of the rotator cuff can be freshened with a rongeur or scalpel which helps prevent pinching of the rotator cuff tendons. Once this is done, the cuff can be repaired utilizing either suture anchors or a transosseous technique.

For the suture anchor technique, the patient's arm is adducted and suture anchors are placed in the greater tuberosity near the lateral articular surface of the humeral head. Once the anchors are placed, a suture passing device, such as the Mitek Expressew®, is used to pass the sutures through the tendon. The sutures are then tied. The access device is then removed and the split in the deltoid can be re-approximated with a figure of 8 suture of size 0 absorbable sutures. The skin is then closed as per the surgeon's preference.

If the transosseous repair is used, the sutures can be passed utilizing a suture grasper/passer such as the Mitek Expressew®. One primary advantage of the transosseous technique is that the sutures, typically a size 2 nonabsorbable suture such as Mitek Orthocord®, can be passed in a modified Mason-Allen fashion to provide a stronger grasping type stitch in the cuff tissue. Once all of the sutures are passed, the deep arm of each of these sutures is passed in a transosseous fashion starting at the junction of the lateral margin of the articular surface of the humeral head and the rotator cuff “footprint” on the greater tuberosity. Typically, this is done with a number 2 trocar or cutting-type needle. Once all of the sutures are passed, they are tied to their corresponding superficial suture arm. As with the suture anchor repair, the access device 634 can now be removed and the split in the deltoid can be re-approximated with a figure of 8 suture of size 0 absorbable suture. The skin is then closed as per the surgeon's preference.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for accessing a surgical site in a patient, said system comprising:
an access device configured for insertion into the surgical site, the access device having a tubular shape with a wall having a wall thickness, a proximal end, a distal end, a channel extending between the proximal and distal ends, and an outer surface circumferentially disposed around the access device,
wherein the outer surface defines a support section and the outer surface also defines an access section having an aperture extending through the wall, and
wherein the channel is sized to receive a surgical instrument, and
wherein the aperture comprises a distal portion and a proximal portion, the distal portion adjacent the distal end of the access device, the proximal portion proximal of the distal portion, and
wherein the channel and the proximal and distal portions of the aperture are sized to receive the surgical instrument, and
wherein the access device is configured to simultaneously retract soft tissue and distract bone when inserted into the surgical site while allowing the surgical instrument access to the surgical site.

2. The system of claim 1, wherein the distal portion has a width and the proximal portion has a width less than the width of the distal portion.

3. The system of claim 1, wherein the wall in the support section extends further distally than the wall in the access section.

4. The system of claim 1, wherein the access device forms a cylindrical tube.

5. The system of claim 1, wherein the access device forms an oval, elliptical, square, diamond, or round tube.

6. The system of claim 1, further comprising one or more tissue dilators sized for slidably positioning in the channel of the access device.

7. The system of claim 6, wherein the one or more tissue dilators comprises a plurality of tissue dilators slidably disposed over one another, the plurality of tissue dilators sized for slidably positioning in the channel of the access device.

8. The system of claim 6, wherein the one or more tissue dilators comprise an expandable member for dilating tissue.

9. The system of claim 1, further comprising an actuatable dilator mechanism having a plurality of arms movably coupled to an outer frame, and an engagement element coupled to each of the arms, wherein actuation of the arms moves the engagement elements inward or outward relative to one another, and wherein the engagement elements retract tissue and distract bone when engaged therewith and actuated.

10. The system of claim 1, wherein the distal end of the access device is chamfered or tapered.

11. The system of claim 1, wherein the access device comprises two separate blades engaged with one another to form the tubular shape.

12. The system of claim 1, further comprising an illumination element coupled with the access device, wherein the illumination element illuminates the surgical site.

13. The system of claim 12, wherein the access device comprises a channel for receiving the illumination element.

14. The system of claim 12, wherein the illumination element comprises an optical waveguide.

15. The system of claim 1, wherein the access device is formed from an optical material such that the access device is also an optical waveguide configured to illuminate the surgical site.

16. The system of claim 1, wherein the access device comprises a flanged region adjacent the proximal end, the flanged region configured to limit insertion depth of the access device into the surgical site.

17. The system of claim 1, wherein the access device comprises one or more suture management features adjacent the proximal end, the suture management features configured to prevent entanglement of sutures.

18. The system of claim 17, wherein the one or more suture management features comprise slotted regions adjacent the proximal end of the access device.

19. The system of claim 1, further comprising one or more sutures.

20. The system of claim 1, wherein the access device further comprises an engagement element for coupling with a table mounted arm or wall mounted arm.

21. The system of claim 1, wherein the outer surface defines an access window disposed in the support section of the access device, the access window sized to receive the surgical instrument thereby providing greater access to the surgical site.

22. The system of claim 1, further comprising an obturator disposed in the channel of the access device.

23. The system of claim further comprising a dilator and a locking mechanism, and wherein the dilator is disposed in the channel and in the obturator, and wherein the locking mechanism releasably locks the obturator with the dilator.

24. The system of claim 1, further comprising a grasping element releasably attached to the access device, the grasping element adapted to facilitate grasping thereof by an operator.

25. A method for accessing a surgical site through an incision, said method comprising:
providing an access device having a proximal end, a distal end, a channel extending therebetween, and an outer surface that defines a support section and an aperture;
inserting the access device into the incision and toward the surgical site;
simultaneously retracting soft tissue and distracting bones adjacent the surgical site;
inserting a surgical instrument into the channel of the access device and advancing the surgical instrument towards the aperture;
treating target tissue adjacent in the surgical site with the surgical instrument while the access device simultaneously retracts soft tissue and distracts bones.

26. The method of claim 25, wherein the surgical site comprises a rotator cuff in a patient's shoulder.

27. The method of claim 25, wherein the target tissue comprises a tendon.

28. The method of claim 25, wherein retracting the soft tissue comprises retracting the deltoid muscle and wherein distracting the bones comprises distracting the acromion away from the humeral head.

29. The method of claim 25, further comprising dilating the incision with one or more increasingly sized tissue dilators slidably disposed over one another.

30. The method of claim 29, wherein dilating the incision comprises slidably inserting the one or more increasingly sized tissue dilators into the channel of the access device.

31. The method of claim 25, further comprising dilating the incision by actuating a dilator mechanism having a plurality of arms movably coupled to an outer frame, and an engagement element coupled to each of the arms, wherein actuation of the arms moves the engagement elements inward or outward relative to one another, and wherein the engagement elements retract tissue and distract bone when engaged therewith and actuated.

32. The method of claim 25, further comprising illuminating the surgical site with light.

33. The method of claim 32, wherein the light is emitted from an optical waveguide coupled with the access device.

34. The method of claim 32, wherein the access device is formed from an optical material such that the access device is also an optical waveguide, and wherein the light is emitted from the optical waveguide.

35. The method of claim 25, wherein the access device comprises a plurality of suture management features adjacent the proximal end thereof, the method further comprising engaging one or more sutures with the suture management features so as to reduce or eliminate suture entanglement.

36. The method of claim 25, wherein the access device comprises two splittable blades, the method further comprising separating the two blades away from one another.

37. The method of claim 25, further comprising manipulating a grasping element coupled with the access device to rotate or move the access device.

38. The method of claim 25, further comprising inserting an obturator in the access device.

39. The method of claim 25, further comprising inserting a dilator into the access device.
